SEQUENCE LISTING (49 sequences)

SEQ ID NO: 1 - protein, 9 aa, Artificial sequence
  Synthetic peptide
EXEXWXRXX

SEQ ID NO: 2 - protein, 9 aa, Artificial sequence
  Synthetic peptide
EVEWWYRSG

SEQ ID NO: 3 - protein, 9 aa, Artificial sequence
  Synthetic peptide
EVEYWYRSG

SEQ ID NO: 4 - protein, 9 aa, Artificial sequence
  Synthetic peptide
EVEWWYRSV

SEQ ID NO: 5 - protein, 9 aa, Artificial sequence
  Synthetic peptide
EMEHWYRSG

SEQ ID NO: 6 - protein, 9 aa, Artificial sequence
  Synthetic peptide
EVEHWYRVG

SEQ ID NO: 7 - protein, 9 aa, Artificial sequence
  Synthetic peptide
EVEYWYRVG

SEQ ID NO: 8 - protein, 9 aa, Artificial sequence
  Synthetic peptide
EVEYWHRSG

SEQ ID NO: 9 - protein, 9 aa, Artificial sequence
  Synthetic peptide
ESEYWYRVG

SEQ ID NO: 10 - protein, 9 aa, Artificial sequence
  Synthetic peptide
ESEWWYRSG

SEQ ID NO: 11 - protein, 9 aa, Artificial sequence
  Synthetic peptide
ESEHWYRSG

SEQ ID NO: 12 - protein, 9 aa, Artificial sequence
  Synthetic peptide
EVEHWYRTG

SEQ ID NO: 13 - protein, 9 aa, Artificial sequence
  Synthetic peptide
EVEHWYRFW

SEQ ID NO: 14 - protein, 9 aa, Artificial sequence
  Synthetic peptide
EVEFWARGP

SEQ ID NO: 15 - protein, 9 aa, Artificial sequence
  Synthetic peptide
EVEMWRREG

SEQ ID NO: 16 - protein, 9 aa, Artificial sequence
  Synthetic peptide
EVERWARSP

SEQ ID NO: 17 - protein, 9 aa, Artificial sequence
  Synthetic peptide
ELEHWLRKG

SEQ ID NO: 18 - protein, 9 aa, Artificial sequence
  Synthetic peptide
EVEHWYRTG

SEQ ID NO: 19 - protein, 9 aa, Artificial sequence
  Synthetic peptide
EVEHWYRFW

SEQ ID NO: 20 - protein, 9 aa, Artificial sequence
  Synthetic peptide
EVEFWARGP

SEQ ID NO: 21 - protein, 9 aa, Artificial sequence
  Synthetic peptide
EVEMWRREG

SEQ ID NO: 22 - protein, 9 aa, Artificial sequence
  Synthetic peptide
EVERWARSP

SEQ ID NO: 23 - protein, 9 aa, Artificial sequence
  Synthetic peptide
EIEHWWRSG

SEQ ID NO: 24 - protein, 9 aa, Artificial sequence
  Synthetic peptide
ETMGVPWSP

SEQ ID NO: 25 - protein, 9 aa, Artificial sequence
  Synthetic peptide
REVDHWLRH

SEQ ID NO: 26 - protein, 9 aa, Artificial sequence
  Synthetic peptide
SKEPSFWNG

SEQ ID NO: 27 - protein, 9 aa, Artificial sequence
  Synthetic peptide
ETMGVPWSP

SEQ ID NO: 28 - protein, 9 aa, Artificial sequence
  Synthetic peptide
TEADHWFRS

SEQ ID NO: 29 - protein, 9 aa, Artificial sequence
  Synthetic peptide
SKEPSFWNG

SEQ ID NO: 30 - protein, 9 aa, Artificial sequence
  Synthetic peptide
EIEHWWRSG

SEQ ID NO: 31 - DNA, 23 bp, Artificial sequence
  Synthetic oligonucleotide
aauuccuggcgagaaggacucnn

SEQ ID NO: 32 - DNA, 23 bp, Artificial sequence
  Synthetic oligonucleotide
nnuuaaggaccgcucuuccugag

SEQ ID NO: 33 - DNA, 20 bp, Artificial sequence
  Synthetic oligonucleotide
cgtgtctcagagaccgactt

SEQ ID NO: 34 - DNA, 15 bp, Artificial
  Synthetic oligonucleotide
atggagggcgccggc

SEQ ID NO: 35 - protein, 12 aa, Artificial sequence
  Synthetic peptide
SPLAQAVRSSSR

SEQ ID NO: 36 - protein, 9 aa, Artificial sequence
  Synthetic peptide
EVEHWYRSG

SEQ ID NO: 37 - protein, 9 aa, Artificial sequence
  Synthetic peptide
AVAHWYRSG

SEQ ID NO: 38 - protein, 9 aa, Artificial sequence
  Synthetic peptide
EVEHAYRSG

SEQ ID NO: 39 - protein, 9 aa, Artificial sequence
  Synthetic peptide
EVEHWARSG

SEQ ID NO: 40 - protein, 9 aa, Artificial sequence
  Synthetic peptide
EVEHWYASG

SEQ ID NO: 41 - protein, 381 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 42 - DNA, 1357 bp, Homo sapiens
gcaactcgctccggccgctgggcgtagctgcgactcggcggagtcccggcggcgcgtccttgttctaacccggcgcgccatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgtcttctatctgggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttcaaaaaaaaaaaaaaaaaaaaaaaaaaaaaa

SEQ ID NO: 43 - protein, 19 aa, Artificial sequence
  Synthetic peptide
DCGLPPDVPNAQPALEGRT

SEQ ID NO: 44 - protein, 19 aa, Artificial sequence
  Synthetic peptide
CEESFVKIPGEKDSVTCLK

SEQ ID NO: 45 - protein, 7 aa, Artificial sequence
  Synthetic peptide
SCEVPTR

SEQ ID NO: 46 - protein, 8 aa, Artificial sequence
  Synthetic peptide
REPSLSPK

SEQ ID NO: 47 - protein, 9 aa, Artificial sequence
  Synthetic peptide
WSTAVEFCK

SEQ ID NO: 48 - protein, 9 aa, Artificial sequence
  Synthetic peptide
SCPNPGEIR

SEQ ID NO: 49 - protein, 25 aa, Artificial sequence
  Synthetic peptide
EIYCPAPPQIDNGIIQGERDHYGYR

(19) United States
(12) Patent Application Publication
(10) Pub. No.: US 2005/0169914 A1
(43) Pub. Date: Aug. 4, 2005

(54) Novel functions for decay accelerating factor (DAF) in inflammation

(76) Inventors: Sean P. Colgan, North Reading, MA (US); Gregory L. Stahl, Clinton, MA (US)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/910,802

(22) Filed: Aug. 2, 2004

Related U.S. Application Data
(60) Provisional application No. 60/492,140, filed on Aug. 1, 2003.

(51) Int. Cl. A61K 39/395; C07H 21/04; C07K 16/42; C12N 5/06

(52) U.S. Cl. 424/131.1; 530/387.2; 435/69.1; 435/327; 435/320.1; 536/23.53

(57) ABSTRACT

The invention involves methods and products for interfering with neutrophil transmigration and treating inflammatory conditions. Agents that bind decay accelerating factor (DAF) and agents that mimic SCR-3 epitope of DAF are provided.

### RELATED APPLICATIONS

This application claims benefit under 35 U.S.C. 119(e) of U.S. provisional patent application 60/492,140, filed Aug. 1, 2003, the entire content of which is incorporated herein by reference.

### FEDERALLY SPONSORED RESEARCH

This work was supported by National Institutes of Health grants HL54229, DK50189 and DE13499 and by a grant from the Crohn's and Colitis Foundation of America (CCFA). The Government may have certain rights to this invention.

### BACKGROUND OF THE INVENTION

Neutrophils (polymorphonuclear leukocytes, PMN) are the first line of host defense against bacterial pathogens. The majority of pathogens are encountered at mucosal surfaces, and identification of pathways which elicit effective PMN mobilization to the epithelium has been an area of active investigation. In order to engage pathogens at sites of inflammation, PMN must first migrate out of circulation through the endothelial cell layer and into host tissue. Infections involving mucosal epithelial surfaces (e.g., lung, oral cavity) require that PMN migrate to epithelium in response to tissue-derived signals. Transepithelial migration of PMN is the pathological hallmark of active mucosal inflammation and occurs in such disease states as inflammatory bowel disease (IBD), periodontitis, cystitis, and infectious enterocolitis. Migration of PMN through epithelial barriers involves a concerted series of cell surface crosstalk events between the PMN and epithelial cells. Evidence exists that initial PMN-epithelial binding requires PMN β₂ integrins including CD11b/18 (Turner et al., 1997, Am. J. Physiol. 273: C1378-85) and that subsequent movement of PMN through the paracellular space is dependent on epithelial CD47 interactions with PMN-expressed signal regulatory protein-alpha (Sirp-α).

A critical site for PMN-pathogen interactions is the apical epithelial membrane, and this membrane domain represents the terminal stage for migrating PMN (Turner et al., 1997, Am. J. Physiol. 273: C1378-85). The histopathological hallmark of many mucosal inflammatory diseases is the formation of crypt abscesses, defined as the migration of large numbers of PMN across the apical epithelial surface and into the lumenal aspect of the tissue. Such observations suggest that PMN utilize apical epithelial ligands to accomplish this task, yet apical ligands have not been studied in detail. Some evidence indicates that under certain circumstances (e.g. inflammatory bowel diseases), intercellular adhesion molecule-1 (ICAM-1) is expressed on the apical epithelial surface and can serve as an apical retention signal for PMN on the epithelium (Dickson et al., 2000, Mol. Cell. Biol. 20: 1436-47). Moreover, recent studies have demonstrated that PMN Fc receptor binding to epithelial-bound antibody may contribute to the localization and retention of PMN on the apical membrane. At present, it is not known what molecular triggers might promote the dislocation of PMN from the epithelial surface.

### SUMMARY OF THE INVENTION

Decay accelerating factor (DAF) is believed to be involved in certain immunological interactions; DAF is known to bind, via CD97, hematologic cells that express CD97. Neutrophils have been characterized as not expressing CD97.

It has been discovered, unexpectedly, that DAF is involved in neutrophil transmigration and that agents that bind to DAF or that mimic DAF can be used to interfere with neutrophil transmigration.

According to one aspect of the invention, there is provided an isolated polypeptide that binds decay accelerating factor (DAF) and competitively inhibits the specific binding of OE-1 antibody to DAF. The OE-1 antibody is an SCR-3 specific ATCC antibody produced by a hybridoma cell line, which was deposited on Jul. 30, 2003 pursuant to, and in satisfaction of, the requirements of the Budapest Treaty on the International Recognition of the Deposit of Microorganisms for the Purposes of Patent Procedure with the American Type Culture Collection (“ATCC”) as an International Depository Authority and given the Patent Deposit Designations PTA-5362. For purposes of brevity the term “deposited hybridoma” or “hybridoma OE-1” or “OE-1” is used throughout the specification to refer to the hybridoma deposited with the ATCC on. The term “deposited monoclonal antibody” or “OE-1 anti-human mAb” or “OE-1 mAb” is used to refer to the monoclonal antibody produced by the ATCC deposited hybridoma.

In one embodiment, the isolated polypeptide binds SCR-3 epitope of DAF. In one important embodiment, the isolated polypeptide is an antibody or antigen binding fragment thereof. The antibody or antigen-binding fragment thereof can interfere with transmigration of neutrophils across a cellular membrane. In one embodiment, the antibody or antigen-binding fragment thereof is selected for its ability to bind living cells. Preferably, the antibody or antigen-binding fragment thereof binds to a conformational epitope of DAF. In one embodiment, the antibody or antigen-binding fragment thereof binds SCR-3 epitope of DAF. The antibody can be a polyclonal or a monoclonal antibody. In one important embodiment, the monoclonal antibody is OE-1.

According to another aspect of the invention, there is provided an isolated polypeptide which has the amino acid sequence EX₁EX₂WX₂R X₁X₃ (SEQ ID NO: 1), wherein X₁ is a neutral amino acid, X₂ is a large amino acid and X₃ is a hydrophobic amino acid. In one embodiment, X₁ is selected from G, A, V, L, I, M, P, F, W, S, T, N, Q, Y, and C; X₂ is selected from W, Y, R, H, and F; and X₃ is selected from G, A, V, L, I, M, P, F, and W. Examples of specific such isolated polypeptides include, but are not limited to, a polypeptide having a sequence selected from the group consisting of: EVEWWYRSG (SEQ ID NO: 2), EVEYWYRSG (SEQ ID NO: 3), EVEWWYRSV (SEQ ID NO: 4), EMEHWYRSG (SEQ ID NO: 5), EVEHWYRVG (SEQ ID NO: 6), EVEYWYRVG (SEQ ID NO: 7), EVEYWHRSG (SEQ ID NO: 8), ESEYWYRVG (SEQ ID NO: 9), ESEWWYRSG (SEQ ID NO: 10), ESEHWYRSG (SEQ ID NO: 11), EVEHWYRTG (SEQ ID NO: 12), EVEHWYRFW (SEQ ID NO: 13), EVEFWARGP (SEQ ID NO: 14), EVEMWRREG (SEQ ID NO: 15), EVERWARSP (SEQ ID NO: 16), ELEHWLRKG (SEQ ID NO: 17), EVEHWYRTG (SEQ ID NO: 18), EVEHWYRFW (SEQ ID NO: 19), EVEFWARGP (SEQ ID NO: 20), EVEMWRREG (SEQ ID NO: 21), EVERWARSP (SEQ ID NO: 22), EIEHWWRSG (SEQ ID NO: 23), ETMGVPWSP (SEQ ID NO: 24), REVDHWLRH (SEQ ID NO: 25), SKEPSFWNG (SEQ ID NO: 26), ETMGVPWSP (SEQ ID NO: 27), TEADHWFRS (SEQ ID NO: 28), SKEPSFWNG (SEQ ID NO: 29) and EIEHWWRSG (SEQ ID NO: 30).

In one embodiment, preferably, the polypeptide interferes with transmigration of neutrophils across a cellular membrane. In one embodiment, the polypeptide binds OE-1 antibody.

According to another aspect of the invention, an isolated polypeptide is provided that mimics a SCR-3 epitope of DAF and interferes with transmigration of neutrophils across a cellular membrane. In one embodiment the isolated polypeptide mimics a conformational epitope of SCR-3. Preferably, the isolated polypeptide binds OE-1 antibody. Examples of such polypeptides include but are not limited to, a polypeptide having a sequence selected from the group consisting of: EVEHWYRTG (SEQ ID NO: 12), EVEHWYRFW (SEQ ID NO: 13), EVEFWARGP (SEQ ID NO: 14), EVEMWRREG (SEQ ID NO: 15), EVERWARSP (SEQ ID NO: 16), ELEHWLRKG (SEQ ID NO: 17), EVEHWYRTG (SEQ ID NO: 18), EVEHWYRFW (SEQ ID NO: 19), EVEFWARGP (SEQ ID NO: 20), EVEMWRREG (SEQ ID NO: 21), EVERWARSP (SEQ ID NO: 22), EIEHWWRSG (SEQ ID NO: 23), ETMGVPWSP (SEQ ID NO: 24), REVDHWLRH (SEQ ID NO: 25), SKEPSFWNG (SEQ ID NO: 26), ETMGVPWSP (SEQ ID NO: 27), TEADHWFRS (SEQ ID NO: 28), SKEPSFWNG (SEQ ID NO: 29) and EIEHWWRSG (SEQ ID NO: 30). In one embodiment, the polypeptide comprises the sequence EVEHWYRSG (SEQ ID NO: 36).

According to another aspect of the invention, both isolated nucleic acids free of a recombinant expression vector and recombinant expression vectors containing such isolated nucleic acids are provided. The recombinant expression vector can contain the isolated nucleic acid operably-linked to a promoter. The isolated nucleic acid and complementary sequences thereof encode any one of the polypeptides discussed herein.

According to another aspect of the invention, a host cell is provided containing the above expression vectors.

According to still another aspect of the invention, pharmaceutical preparations are provided. The pharmaceutical preparations contain, for example, an isolated polypeptide of the invention or an isolated nucleic acid of the invention and a pharmaceutically acceptable carrier. The pharmaceutical preparation optionally can contain another pharmaceutical agent in addition to the compounds of the invention discussed herein. In certain embodiments, the agent is a nonsteroidal anti-inflammatory agent. In other important embodiments, the anti-inflammatory agent is a corticosteroid, a COX inhibitor or a tumor necrosis factor antagonist. In still other embodiments, the other agent is an anti-bacterial agent, an antifungal agent or a drug for treating inflammatory bowel disease.

According to another aspect of the invention, a method is provided for modulating an immunological interaction. The method involves contacting cells bearing DAF with an agent that binds DAF and that interferes with the interaction between neutrophils and the cells bearing DAF. The agent can be any one of such agents described herein. In an important embodiment, the agent that binds DAF is an antibody or antigen binding fragment thereof, such as an OE-1 antibody.

According to another aspect of the invention, a method is provided for modulating an immunological interaction by contacting neutrophils with an agent that mimics the SCR-3 epitope of DAF and that interferes with the interaction between the neutrophils and cells bearing DAF. The agent can be any such agent as described herein.

According to another aspect of the invention, a method is provided for treating inflammation. The method involves administering to a subject in need of such treatment an effective amount of an agent that binds DAF or mimics SCR-3 epitope of DAF, wherein the agent interferes with the interaction between neutrophils and DAF bearing cells. The agent can be as described herein. In one embodiment, the inflammation is mucosal inflammation.

According to another aspect of the invention, a method is provided for interfering with neutrophil transmigration by contacting a cell barrier across which neutrophils transmigrate and which includes cells expressing DAF, with an agent that binds DAF. The agent is as described herein.

According to another aspect of the invention, a method is provided for interfering with neutrophil transmigration by contacting neutrophils with an agent that mimics the SCR-3 epitope of DAF and that interferes with the interaction between neutrophils and endothelial or epithelial cells bearing DAF. The agents are as described herein.

According to another aspect of the invention, a method is provided for identifying an agent that interferes with transmigration of neutrophils. The method includes contacting a membrane across which the neutrophils transmigrate with an agent that is a candidate to bind DAF, contacting the membrane with the neutrophils, and determining whether the neutrophils transmigrate across the membrane. In one embodiment, the membrane is polarized. In another embodiment, the membrane is an epithelial monolayer. In another embodiments, the membrane is an endothelial monolayer. In important embodiments, the agent is a polypeptide.

In another aspect of the invention, a method is provided for identifying an agent that interferes with transmigration of neutrophils. This method involves contacting the neutrophils with an agent that is a candidate to mimic the SCR-3 epitope of DAF, contacting a membrane across which leukocytes transmigrate with the neutrophils, and determining whether the neutrophils transmigrate across the membrane. The cell membranes can comprise an epithelial monolayer or an endothelial monolayer. The membranes can be polarized. In important embodiments, the agent is a polypeptide.

In another aspect of the invention, a method is provided for treating inflammation by modulating the cellular levels of hypoxin inducible factor (HIF) levels. In certain embodiments of the invention a method is provided for treating conditions that involve unwanted transmigration of neutrophils including hypoxic conditions and ulcers by modulation of the cellular levels of hypoxin inducible factor (HIF). In one aspect of the invention a method is provided for treating inflammation by decreasing or inhibiting the levels of cellular activity of HIF (e.g. using antibody, antisense, or siRNA technology, or using one or more other biological or synthetic inhibitors of HIF activity or expression). Hypoxic conditions include but are not limited to hypoxia resulting from diabetic ischemia, pulmonary hypertension, reperfusion injury, hypoxia resulting from cardiovascular diseases including myocardial infarction, surgery, stroke, arthritis, sepsis and the like.

In another aspect of the invention a method for screening and diagnosis based on single nucleotide polymorphism analysis of the DAF promoter region described herein. In addition the invention encompasses methods of modulating the activity of the DAF promoter and thereby treating inflammatory diseases and hypoxic conditions described herein.

### BRIEF DESCRIPTION OF DRAWINGS

FIG. 1 is a bar graph that shows the functional influence of OE-1 antibody on transmigration.

FIG. 2 shows the localization of OE-1 antigen to the apical surface.

FIG. 3 illustrates the biochemical identification of OE-1 antigen as human CD55; decay-accelerating factor (DAF). OE-1 and DAF peptides are shown: DCGLPPDVPNAQPALEGRT (SEQ ID NO: 43), CEESFVKIPGEKDSVTCLK (SEQ ID NO: 44), SCEVPTR (SEQ ID NO: 45), REPSLSPK (SEQ ID NO: 46), WSTAVEFCK (SEQ ID NO: 47), SCPNPGEIR (SEQ ID NO: 48), and EIYCPAPPQIDNGIIQGERDHYGYR (SEQ ID NO: 49).

FIG. 4 is a set of graphs that show the role of DAF in the kinetics of PMN transmigration.

FIG. 5 illustrates the influence of DAF depletion on PMN transmigration.

FIG. 6 shows the SCR domain mapping and identification of OE-1 epitope in Panel A. Panel B shows peptide sequences EVEHWYRTG (SEQ ID NO: 12), EVEHWYRFW (SEQ ID NO: 13), EVEFWARGP (SEQ ID NO: 14), EVEMWRREG (SEQ ID NO: 15), EVERWARSP (SEQ ID NO: 16), ELEHWLRKG (SEQ ID NO: 17), EIEHWWRSG (SEQ ID NO: 23), ETMGVPWSP (SEQ ID NO: 24), REVDHWLRH (SEQ ID NO: 25), SKEPSFWNG (SEQ ID NO: 26), ETMGVPWSP (SEQ ID NO: 27), TEADHWFRS (SEQ ID NO: 28), SKEPSFWNG (SEQ ID NO: 29), and a consensus sequence EVEHWYRSG (SEQ ID NO: 36). Panels C and D show experiments using EVEHWYRSG (SEQ ID NO: 36).

FIG. 7 is a graph that shows the influence of OE-1 peptide mimetic on the kinetics of PMN transmigration. Panels A and B show experiments using EVEHWYRSG (SEQ ID NO: 36).

FIG. 8 is a graph that shows the influence of OE-1 peptide mimetics on the kinetics of PMN transmigration.

FIG. 9 shows that epithelial hypoxia in TNBS colitis is associated with inflammatory lesions.

FIG. 10 is a graph that shows that epithelial hypoxia increases the rate of PMN transmigration.

FIG. 11 is a graph that shows the influence of hypoxia on CD55 mRNA and protein expression in cultured intestinal epithelial cells.

FIG. 12 is a graph that shows the results from CD55 gene promoter luciferase reporter assays.

FIG. 13 is a graph that shows that the over-expression of epithelial CD55 functionally mimics hypoxia-increased PMN transmigration.

### DETAILED DESCRIPTION OF THE INVENTION

Decay accelerating factor (DAF) is a highly glycosylated, 70-80 kDa, glycosylphosphatidyl inositol (GPI)-anchored protein. It is a member of the cell membrane bound complement regulatory proteins that function as inhibitors of autologous complement lysis by inhibiting C3/C5 convertases. DAF is expressed on cells that are in close contact with serum complement proteins and on cells outside the vascular space and on tumor cells. DAF also is expressed on the apical membrane in polarized epithelial cells. DAF is organized in four homologous short consensus repeats (SCR, also called complement control protein repeats). The SCR domains play specific roles in DAF cellular interactions.

Neutrophil migration through cell membranes (barriers formed by one or more layers of cells) is a first stage of inflammation. Such migration can be unwanted or excessive and itself can contribute to disease. This is particularly the case with mucosal inflammatory disease involving transmigration of large numbers of neutrophils through cell membranes and release into the luminal aspects of tissue.

The invention provides methods and products for interfering with neutrophil transmigration. These methods and products can be applied in research settings to better understand the inflammatory process and to influence the movement and/or accumulation of neutrophils. They also can be applied in medical settings to reduce unwanted inflammation, and, in important embodiments, to treat inflammatory conditions including mucosal inflammatory conditions. Mucosal inflammatory conditions include inflammatory bowel disease, gingivitis, periodontitis, psoriasis, conjunctivitis, and bacterial infection of mucosal surfaces including pneumonia. Inflammatory bowel diseases are diseases such as ulcerative colitis, Crohn's disease and the like.

Other conditions involving unwanted transmigration of neutrophils include hypoxic conditions and ulcers. Hypoxic conditions include but are not limited to hypoxia resulting from diabetic ischemia, pulmonary hypertension, reperfusion injury, hypoxia resulting from cardiovascular diseases including myocardial infarction, surgery, stroke, arthritis, sepsis and the like.

The invention thus involves methods for modulating an immunological interaction. By modulating an immunological interaction, it is meant altering the interaction between neutrophils and cells bearing DAF. Most typically, the modulation will involve interfering with that interaction. It is also possible according to the invention, however, to promote such interaction by upregulating the expression of DAF through stimulation of endogenous DAF expression or through introducing recombinant DAF into cells, as will be recognized by those of ordinary skill in the art according to known methods.

The methods for interfering with neutrophil transmigration include blocking the interaction between a neutrophil and a DAF bearing (expressing) cell. In one important embodiment, this occurs at the endothelial cell membrane created by a layer of endothelial cells. These methods also include interfering with the release of neutrophils from the apical epithelial cell membrane created by a layer of epithelial cells defining, for example, the mucosal surfaces of the body including the lungs, the oral cavity, the gastrointestinal tract, the vagina and the like. Neutrophil transmigration can be assessed in vitro or in vivo by methods well known to those of ordinary skill in the art, directly or indirectly.

The invention also involves methods for treating inflammation in a subject by interfering with the interaction between neutrophils and DAF bearing cells. In important embodiments, inflammation is treated by inhibiting, reducing or preventing in whole or in part neutrophil transmigration in a subject. Subjects include humans, non-human primates, dogs, cats, sheep, goats, horses, pigs, cows, rodents, worms, fish and frogs.

In carrying out the various methods of the invention, two preferred types of agents are employed. One preferred type of agent can be those which bind DAF and interfere with the interaction between neutrophils and cells bearing DAF. Another preferred type of agents can be those which mimic DAF and interfere with the interaction between neutrophils and cells bearing DAF, presumably by binding to the DAF binding-partner on neutrophils. Using methods according to the invention for identifying such agents, about thirty such agents have been identified which are effective for the methods of the invention.

Thus, the invention also involves methods for identifying agents which interfere with neutrophil transmigration. In one aspect, a cellular membrane across which neutrophils transmigrate is contacted with an agent that a candidate to bind DAF. The cellular membrane then is contacted with neutrophils. It then is determined whether and/or to what extent the neutrophils transmigrate at the cellular membrane. The method can be performed in vitro or in vivo. If performed in vivo, native neutrophils simply are permitted to contact the cellular membrane. If performed in vitro, neutrophils would, of course, be supplied. In one important embodiment, the cellular membrane comprises a layer of epithelial cells. In another important embodiment, the cellular membrane comprises a layer of endothelial cells. The membrane in important embodiments is polarized. The membrane preferably is a membrane through which neutrophils migrate in vivo.

In another aspect, an agent that mimics DAF is contacted with neutrophils. The neutrophils are contacted with or are already in contact with a cellular membrane. It is determined whether, and/or to what extent, the neutrophils transmigrate at the cellular membrane. As used herein, transmigrate means any one of the attachment to, the migration across or the release from a cellular membrane. The end result, of course, would typically be the same when transmigration is interfered with. That is, fewer neutrophils released on the apical side of a membrane than would be the case for a control.

The agents can be any molecule, but preferably an agent is an organic molecule that is nontoxic at doses useful for in vivo therapeutic use. In the case of agents that interfere with neutrophil-DAF interactions, the molecule can be, for example, a synthetic small molecule or can be biologically produced. Examples of each are described below. In the case of synthetic small molecules, the agents can be screened from combinatorial libraries commercially available or otherwise. Small molecules also can be synthetic peptides, such as those which mimic a SCR-3 epitope of DAF or those which bind to a SCR-3 epitope of DAF (e.g., mimic the OE-1 antibody). Small molecules also can be antisense molecules with natural or modified backbones, all of which are well known to those of ordinary skill in the art. The anti-sense molecules can be anti-DAF nucleic acid molecules, used to down regulate DAF expression and thereby interfere with neutrophil-DAF interaction. The nucleic acid encoding DAF is described in numerous instances in the literature, one of which is shown in SEQ ID NO: 42 (human DAF). Agents of the invention also can be biologically produced, such as antibodies and antigen binding fragments which bind DAF (SEQ ID NO: 41), fragments of DAF, peptides that mimic a DAF epitope, antisense oligonucleotides, siRNA, aptamers and the like. For example, an antisense molecule can be an oligonucleotide of between 5-100, preferably 10-50, preferably about 30 nucleotides in length, and complementary to a portion of the mRNA sequence (including the coding sequence) of SEQ ID NO: 42. An siRNA molecule is preferably a double-stranded RNA molecule with a 19 base pair double-stranded region (corresponding to a sequence on the target gene or mRNA) with a 2 base overhang at both ends (preferably a TT dimer overhang at each end). Such molecules are described in greater detail herein. In the case of peptides that mimic DAF, libraries of such peptides can be generated for example through phage display. A number of such peptides have been generated and tested successfully in the examples herein.

As mentioned above, one aspect of the invention is an agent that mimics DAF and interferes with the interaction between neutrophils and cells expressing DAF on their surface. In one important embodiment, the agent mimics a SCR-3 epitope of DAF. In another embodiment, the agent is soluble DAF or a fragment of DAF. In another important embodiment the agent is an anti-isotype, anti-allotype or an anti-idiotype antibody or antigen binding fragment thereof to OE-1 antibody described herein.

The invention further provides a hybridoma cell line, which produces the OE-1 monoclonal antibody. This hybridoma was deposited on Jul. 30, 2003 pursuant to, and in satisfaction of, the requirements of the Budapest Treaty on the International Recognition of the Deposit of Microorganisms for the Purposes of Patent Procedure with the American Type Culture Collection (“ATCC”) as an International Depository Authority and given the Patent Deposit Designations PTA-5362. For purposes of brevity the term “deposited hybridoma” or “hybridoma OE-1” or “OE-1” is used throughout the specification to refer to the hybridoma deposited with the ATCC. The term “deposited monoclonal antibody” or “OE-1 anti-human mAb” or “OE-1 mAb” is used to refer to the monoclonal antibody produced by the ATCC deposited hybridoma.

A preferred set of agents includes peptides that comprise any fragment or multimer of the formula: X₄X₁X₄X₂X₂X₂X₅X₁X₃, wherein X₁ is a neutral amino acid, X₂ is a large amino acid, X₃ is a hydrophobic amino acid, X₄ is a negatively charged amino acid, and X₅ is a positively charged amino acid. X₁ can be selected from G, A, V, L, I, M, P, F, W, S, T, N, Q, Y, and C; X₂ can beselected from W, Y, R, H, and F; X₃ can be selected from G, A, V, L, I, M, P, F, and W; X₄ can be selected from E and D; and X₅ can be selected from R, K and H. The fragments can have the size of about 3, or about 4, or about 5, or about 6, or about 7, or about 8 or more amino acid residues. One preferred agent of the invention has a length of 9 amino acids. The multimers or other peptides that comprise the sequence can have a total length of about ten, or about 20, or about 50, or about 100, or about 200, or about 500 or more amino acid residues.

Another preferred set of agents is described by the formula: EX₁EX₂WX₂R X₁X₃, (SEQ ID NO: 1) wherein X₁ is a neutral amino acid, X₂ is a large amino acid and X₃ is a hydrophobic amino acid. X₁ can be selected from G, A, V, L, I, M, P, F, W, S, T, N, Q, Y, and C; X₂ is selected from W, Y, R, H, and F; and X₃ is selected from G, A, V, L, I, M, P, F, and W. Examples include a polypeptide, wherein the polypeptide has a sequence selected from the group consisting of: EVEWWYRSG (SEQ ID NO: 2), EVEYWYRSG (SEQ ID NO: 3), EVEWWYRSV (SEQ ID NO: 4), EMEHWYRSG (SEQ ID NO: 5), EVEHWYRVG (SEQ ID NO: 6), EVEYWYRVG (SEQ ID NO: 7), EVEYWHRSG (SEQ ID NO: 8), ESEYWYRVG (SEQ ID NO: 9), ESEWWYRSG (SEQ ID NO: 10), ESEHWYRSG (SEQ ID NO: 11), EVEHWYRTG (SEQ ID NO: 12), EVEHWYRFW (SEQ ID NO: 13), EVEFWARGP (SEQ ID NO: 14), EVEMWRREG (SEQ ID NO: 15), EVERWARSP (SEQ ID NO: 16), ELEHWLRKG (SEQ ID NO: 17), EVEHWYRTG (SEQ ID NO: 18), EVEHWYRFW (SEQ ID NO: 19), EVEFWARGP (SEQ ID NO: 20), EVEMWRREG (SEQ ID NO: 21), EVERWARSP (SEQ ID NO: 22), EIEHWWRSG (SEQ ID NO: 23), ETMGVPWSP (SEQ ID NO: 24), REVDHWLRH (SEQ ID NO: 25), SKEPSFWNG (SEQ ID NO: 26), ETMGVPWSP (SEQ ID NO: 27), TEADHWFRS (SEQ ID NO: 28), SKEPSFWNG (SEQ ID NO: 29) and EIEHWWRSG (SEQ ID NO: 30).

This preferred set of agents also includes any peptides that comprise any fragments or multimers of the aforedescribed formula. The fragments can have the size of about 3, or about 4, or about 5, or about 6, or about 7, or about 8 or more amino acid residues. One preferred agent of the invention has a length of 9 amino acids. Preferred fragments are truncated by 1, 2, 3, 4, 5, or 6 amino acids at the C-terminus, N-terminus, internally, or a combination of the above. Any of the peptides described herein (including the 9mers) can be part of a longer peptide with zero, one, or more amino acids independently added at each of the C-terminus, N-terminus, and/or internally. Longer peptides can include two or more full length or truncated peptides described herein, with or without additional amino acid extensions or spacers. A longer peptide can include two or more of copies of any one of the peptides described herein. Alternatively, the longer peptides can include one copy each of two or more different peptides described herein. In further embodiments, the longer peptides can include a combination of the above (e.g. two or more copies of at least one peptide and one copy of at least one other peptide described herein). The multimers or other peptides that comprise the sequence can have a total length of about ten, or about 20, or about 50, or about 100, or about 200, or about 500 or more amino acid residues.

A most preferred such agent is a polypeptide having the sequence EVEHWYRSG (SEQ ID NO: 36).

It will be understood that the foregoing polypeptides, fragments and multimers thereof can be used in various combinations and still be within the scope of the invention. For example two or more of the foregoing peptides can be used together according to the invention.

It will be understood that the foregoing polypeptides can have various modifications made thereto and still be within the scope of the invention. Thus, synthetic versions of polypeptides with non-natural backbones and other modified amino acids known to those of ordinary skill in the art may be substituted for any one or more particular amino acids, provided function as required herein is preserved. For example, each of X₁, X₂, X₃, X₄, and X₅ independently can be a modified (e.g. a rare or non-natural) amino acid, provided that X₁ is neutral, X₂ is large, X₃ is hydrophobic, X₄ is negative, and X₅ is positive.

The invention thus provides novel isolated polypeptides. As used herein with respect to polypeptides, “isolated” means separated from its native environment and present in sufficient quantity to permit its identification or use according to the methods described herein. Isolated, when referring to a protein or polypeptide, means, for example: (i) selectively produced by expression cloning or (ii) purified as by chromatography, electrophoresis or other purification method. Isolated proteins or polypeptides may be, but need not be, substantially pure. The term “substantially pure” means that the proteins or polypeptides are essentially free of other substances with which they may be found in nature or in vivo systems to an extent practical and appropriate for their intended use. Substantially pure polypeptides may be produced by techniques well known in the art. Because an isolated protein may be admixed with a pharmaceutically acceptable carrier in a pharmaceutical preparation, the protein may comprise only a small percentage by weight of the preparation. The protein is nonetheless isolated in that it has been separated from the substances with which it may be associated in living systems, i.e. isolated from other proteins.

The invention also provides agents that are antibodies. As used herein, the term “antibody” refers to a glycoprotein comprising at least two heavy (H) chains and two light (L) chains inter-connected by disulfide bonds. Each heavy chain is comprised of a heavy chain variable region (abbreviated herein as HCVR or V_{H}) and a heavy chain constant region. The heavy chain constant region is comprised of three domains, C_{H}1, C_{H}2 and C_{H}3. Each light chain is comprised of a light chain variable region (abbreviated herein as LCVR or V_{L}) and a light chain constant region. The light chain constant region is comprised of one domain, CL. The V_{H} and V_{L} regions can be further subdivided into regions of hyper variability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “specific binding” refers to antibody binding to a predetermined antigen. Typically, the antibody binds with an affinity that is at least two-fold greater than its affinity for binding to a non-specific antigen (e.g., BSA, casein) other than the predetermined antigen or a closely-related antigen. An isolated antibody that specifically binds to an epitope, isoform or variant of DAF may have cross-reactivity to other related antigens, e.g., from other species (e.g., DAF species homologs).

Accordingly, useful antibodies and peptides of the inventions preferably bind to a DAF epitope, preferably an OE-1 epitope, preferably a SCR-3 epitope with a dissociation constant, Kd of between about 1 μM and about 1 nM (e.g. about 1 nM, about 10, or about 100 nM). This interaction can also be expressed as a specific association constant or affinity, Ka=1/Kd. However, the dissociation constant can be lower or higher. In some embodiments, an antibody can have a dissociation constant lower than 1 nM.

The isolated antibodies of the invention encompass various antibody isotypes, such as IgG1, IgG2, IgG3, IgG4, IgM, IgA1, IgA2, IgAsec, IgD, IgE. As used herein, “isotype” refers to the antibody class (e.g. IgM or IgG1) that is encoded by heavy chain constant region genes. The antibodies can be full length or can include only an antigen-binding fragment such as the antibody constant and/or variable domain of IgG1, IgG2, IgG3, IgG4, IgM, IgA1, IgA2, IgAsec, IgD or IgE or could consist of a Fab fragment, a F(ab′)₂ fragment, and a Fv fragment.

The antibodies of the present invention can be polyclonal, monoclonal, or a mixture of polyclonal, monoclonal or recombinant antibodies. The antibodies can be produced by a variety of techniques well known in the art. Procedures for raising polyclonal antibodies are well known. For example, anti-DAF polyclonal antibodies are raised by administering DAF protein subcutaneously to New Zealand white rabbits which have first been bled to obtain pre-immune serum. The DAF can be injected at a total volume of 100 μl per site at six different sites, typically with one or more adjustments. The rabbits are then bled two weeks after the first injection and periodically boosted with the same antigen three times every six weeks. A sample of serum is collected 10 days after each boost. Polyclonal antibodies are recovered from the serum, preferably by affinity chromatography using DAF to capture the antibody. This and other procedures for raising polyclonal antibodies are disclosed in E. Harlow, et. al., editors, Antibodies: A Laboratory Manual (1988), which is hereby incorporated by reference.

The term “antigen-binding fragment” of an antibody as used herein, refers to one or more portions of an antibody that retain the ability to specifically bind to an antigen (e.g., DAF). It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., 1989 Nature 341:544-546) which consists of a V_{H} domain; and (vi) an isolated complementarity determining region (CDR). Furthermore, although the two domains of the Fv fragment, V and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., 1988, Science 242:423-426; and Huston et al., 1988, Proc. Natl. Acad. Sci. USA 85: 5879-5883). Such single chain antibodies are also intended to be encompassed within the term “antigen-binding portion” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983), which is hereby incorporated by reference as well as by other techniques known to those with skill in the art. The fragments are screened for utility in the same manner as are intact antibodies.

The antibody or antigen-binding fragment thereof preferably is selected for its ability to bind live cells expressing DAF. In order to demonstrate binding of monoclonal antibodies to live cells expressing the DAF, flow cytometry can be used. For example, cell lines expressing DAF (grown under standard growth conditions) are mixed with various concentrations of monoclonal antibodies in PBS containing 0.1% Tween 80 and 20% mouse serum, and incubated at 37° C. for 1 hour. After washing, the cells are reacted with fluorescein-labeled anti-human IgG secondary antibody (if human anti-DAF antibodies were used) under the same conditions as the primary antibody staining. The samples can be analyzed by a fluorescence activated cell sorter (FACS) instrument using light and side scatter properties to gate on single cells. An alternative assay using fluorescence microscopy may be used (in addition to or instead of) the flow cytometry assay. Cells can be stained exactly as described above and examined by fluorescence microscopy. This method allows visualization of individual cells, but may have diminished sensitivity depending on the density of the antigen.

Antibodies also can be selected, for example, based on one or more of the following criteria, which are not intended to be exclusive:
1) high affinity binding to DAF or OE-1 antibody;
2) binding to a SCR-3 epitope on DAF;
3) anti-isotype, anti-allotype or anti-idiotype binding to OE-1 antibody;
4) inhibition in vitro of neutrophil transmigration at a layer of cells expressing DAF.

Preferred antibodies of the invention meet one or more of the foregoing criteria. Antibodies which bind to DAF also can be tested in an in vivo model (e.g., in mice) to determine their efficacy in mediating neutrophil transmigration.

Monoclonal antibody production may be effected by techniques which are also well known in the art. The term “monoclonal antibody,” as used herein, refers to a preparation of antibody molecules of single molecular composition. A monoclonal antibody displays a single binding specificity and affinity for a particular epitope. The process of monoclonal antibody production involves obtaining immune somatic cells with the potential for producing antibody, in particular B lymphocytes, which have been previously immunized with the antigen of interest either in vivo or in vitro and that are suitable for fusion with a B-cell myeloma line.

Mammalian lymphocytes typically are immunized by in vivo immunization of the animal (e.g., a mouse) with the desired protein or polypeptide, e.g., with DAF in the present invention. Such immunizations are repeated as necessary at intervals of up to several weeks to obtain a sufficient titer of antibodies. Once immunized, animals can be used as a source of antibody-producing lymphocytes. Following the last antigen boost, the animals are sacrificed and spleen cells removed. Mouse lymphocytes give a higher percentage of stable fusions with the mouse myeloma lines described herein. Of these, the BALB/c mouse is preferred. However, other mouse strains, rabbit, hamster, sheep and frog may also be used as hosts for preparing antibody-producing cells. See Goding in Monoclonal Antibodies: Principles and Practice, 2d ed., pp. 60-61, Orlando, Fla., Academic Press, 1986.

Those antibody-producing cells that are in the dividing plasmoblast stage fuse preferentially. Somatic cells may be obtained from the lymph nodes, spleens and peripheral blood of antigen-primed animals, and the lymphatic cells of choice depend to a large extent on their empirical usefulness in the particular fusion system. The antibody-secreting lymphocytes are then fused with (mouse) B cell myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, 1975, Nature 256:495, which is hereby incorporated by reference.

Myeloma cell lines suited for use in hybridoma-producing fusion procedures preferably are non-antibody-producing, have high fusion efficiency, and enzyme deficiencies that render them incapable of growing in certain selective media which support the growth of the desired hybridomas. Examples of such myeloma cell lines that may be used for the production of fused cell lines include P3-X63/Ag8, X63-Ag8.653, NS1/1.Ag 4.1, Sp2/0-Ag14, FO, NSO/U, MPC-11, MPC11-X45-GTG 1.7, S194/5XX0 Bul, all derived from mice; R210.RCY3, Y3-Ag 1.2.3, IR983F and 4B210 derived from rats and U-266, GM1500-GRG2, LICR-LON-HMy2, UC729-6, all derived from humans (Goding, in Monoclonal Antibodies: Principles and Practice, 2d ed., pp. 65-66, Orlando, Fla., Academic Press, 1986; Campbell, in Monoclonal Antibody Technology, Laboratory Techniques in Biochemistry and Molecular Biology Vol. 13, Burden and Von Knippenberg, eds. pp. 75-83, Amsterdam, Elseview, 1984).

Fusion with mammalian myeloma cells or other fusion partners capable of replicating indefinitely in cell culture is effected by standard and well-known techniques, for example, by using polyethylene glycol (“PEG”) or other fusing agents (See Milstein and Kohler, 1976, Eur. J. Immunol. 6:511, which is hereby incorporated by reference).

In other embodiments, the antibodies can be recombinant antibodies. The term “recombinant antibody”, as used herein, is intended to include antibodies that are prepared, expressed, created or isolated by recombinant means, such as antibodies isolated from an animal (e.g., a mouse) that is transgenic for another species' immunoglobulin genes, antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

In yet other embodiments, the antibodies can be chimeric or humanized antibodies. As used herein, the term “chimeric antibody” refers to an antibody, that combines the murine variable or hypervariable regions with the human constant region or constant and variable framework regions. As used herein, the term “humanized antibody” refers to an antibody that retains only the antigen-binding CDRs from the parent antibody in association with human framework regions (see, Waldmann, 1991, Science 252:1657). Such chimeric or humanized antibodies retaining binding specificity of the murine antibody are expected to have reduced immunogenicity when administered in vivo for diagnostic, prophylactic or therapeutic applications in humans according to the invention.

In certain embodiments, the antibodies thus are human antibodies. The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse have been grafted onto human framework sequences (referred to herein as “humanized antibodies”). Human antibodies directed against DAF are generated using transgenic mice carrying parts of the human immune system rather than the mouse system.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals results in the production of fully human antibodies to the antigen of interest. Following immunization of these mice (e.g., XenoMouse (Abgenix), HuMAb mice (Medarex/GenPharm)), monoclonal antibodies are prepared according to standard hybridoma technology. These monoclonal antibodies have human immunoglobulin amino acid sequences and therefore will not provoke human anti-mouse antibody (HAMA) responses when administered to humans.

Preferably, the mice are 6-16 weeks of age upon the first immunization. For example, a purified or enriched preparation of DAF antigen (e.g., recombinant DAF or DAF-expressing cells) or OE-1 antibody is used to immunize the mice intraperitoneally (IP), although other routes of immunization known to one of ordinary skill in the art are also possible. DAF antigen is injected in combination with an adjuvant, such as complete Freund's adjuvant, and preferably the initial injection is followed by booster immunizations with antigen in an adjuvant, such as incomplete Freund's adjuvant. The immune response is monitored over the course of the immunization protocol with plasma samples obtained by, for example, retroorbital bleeds. The plasma is screened by ELISA (as described below), and mice with sufficient titers of anti-DAF human immunoglobulin are used for fusions. Mice are boosted intravenously with antigen 3 days before sacrifice and removal of the spleen.

In one important aspect of the invention, the agents bind DAF and interfere with neutrophil transmigration. In a particularly important aspect of the invention, the agent binds an SRC-3 conformational epitope of DAF. The agent then is capable of binding, and can be selected for its ability to bind, living cells. Thus, in one aspect of the invention, the agent binds to a conformational epitope within the extracellular domain of the DAF molecule. To determine if an agent binds to conformational epitopes, each agent can be tested in assays using native protein (e.g., non-denaturing immunoprecipitation, flow cytometric analysis of cell surface binding) and denatured protein (e.g., Western blot, immunoprecipitation of denatured proteins). A comparison of the results will indicate whether the agent binds conformational epitopes.

As described above, the agent can be a polypeptide. One such polypeptide is an antibody that binds a DAF bearing cell or an antigen binding fragment thereof. A particularly preferred antibody is OE-1 antibody. This antibody is produced by the hybridoma cell line OE-1 described above and deposited with the ATCC, PTA-5362. ATCC stands for the American Type Culture Collection (Rockville, Md., USA) an International Depository Authority under the Budapest Treaty on the International Recognition of the Deposit of Microorganisms for the Purposes of Patent Procedure.

The invention also embraces the deposited hybridoma cell line and the nucleic acid it contains which encodes the OE-1 antibody. The OE-1 antibody was selected by a screen of a panel of antibodies raised against epithelial plasma membranes. It was subsequently discovered that OE-1 antibody binds DAF, and it is believed that OE-1 antibody binds a confoniational epitope of DAF, SCR-3. OE-1 antibody and antigen binding fragments thereof are useful to bind DAF and block neutrophil interaction with DAF. OE-1 antibodies also appear to mimic a cell surface component of neutrophils and can be used to select peptides which mimic a SCR-3 epitope of DAF and which, apparently, bind to neutrophils and block neutrophil-DAF interactions.

The invention also involves agents, including polypeptides, antibodies and antigen binding fragments thereof, which competitively inhibit OE-1 antibodies to DAF. Thus, agents useful according to the invention include those which competitively inhibit the specific binding of an OE-1 antibody to its target epitope on DAF. To determine competitive inhibition, a variety of assays known to one of ordinary skill in the art can be employed. Competition assays, for example, can be used to determine if an agent competitively inhibits OE-1 antibody from binding to DAF. Competition assays include cell-based methods such as flow cytometry and solid phase binding assays. Important embodiments include measuring the decrease in binding of OE-1 to living cells expressing DAF in the presence of increasing concentrations of a competitive inhibitor of OE-1 antibody.

Certain preferred agents competitively inhibit the specific binding of OE-1 antibody to its target epitope on DAF by at least about 10%, 20%, 30%, 40%, 50%, 60%, 70%, 80%, 90%, 95%, or 99%. Inhibition can be assessed at various molar ratios or mass ratios; for example competitive binding experiments can be conducted with a 2-fold, 3-fold, 4-fold, 5-fold, 7-fold, 10-fold or more molar excess of the agent versus OE-1 antibody.

The invention also includes novel nucleic acids encoding the above polypeptides. These novel nucleic acids are those encoding the polypeptides of the formula X₄X₁X₄X₂X₂X₂X₅X₁X₃, wherein X₁ is a neutral amino acid, X₂ is a large amino acid and X₃ is a hydrophobic amino acid. X₁ can be selected from G, A, V, L, I, M, P, F, W, S, T, N, Q, Y, and C; X₂ is selected from W, Y, R, H, and F; and X₃ is selected from G, A, V, L, I, M, P, F, and W; X₄ is E or D; and X₅ is R, K or H; preferably of the formula EX₁EX₂WX₂R X₁X₃ (SEQ ID NO: 1), wherein X₁ is a neutral amino acid, X₂ is a large amino acid and X₃ is a hydrophobic amino acid. X₁ can be selected from G, A, V, L, I, M, P, F, W, S, T, N, Q, Y, and C; X₂ is selected from W, Y, R, H, and F; and X₃ is selected from G, A, V, L, I, M, P, F, and W; including fragemnt or multimers thereof, and nucleic acids encoding the specific examples of such polypeptides described herein, and nucleic acids encoding OE-1 antibody and antigen binding fragments thereof.

The nucleic acid molecules of the invention are isolated. As used herein with respect to nucleic acid molecules, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid molecule is one which is readily manipulated by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid molecule may be substantially purified, but need not be. For example, a nucleic acid molecule that is isolated within a cloning or expression vector is not pure in that it may comprise only a tiny percentage of the material in the cell in which it resides. Such a nucleic acid molecule is isolated, however, as the term is used herein because it is readily manipulated by standard techniques known to those of ordinary skill in the art. An isolated nucleic acid molecule as used herein is not a naturally occurring chromosome.

The term complementary nucleic acid sequences means nucleic acid sequences that can form a perfect base-paired double helix with each other.

The nucleic acid molecules of the invention also include degenerate nucleic acid molecules which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating polypeptide. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acid molecules that differ from the biologically isolated nucleic acids in codon sequence due to the degeneracy of the genetic code.

The invention also provides modified nucleic acid molecules which encode the antibodies or antigen binding fragments described herein, including additions, substitutions and deletions of one or more nucleotides. In preferred embodiments, these modified nucleic acid molecules and/or the polypeptides they encode retain at least one activity or function of the unmodified nucleic acid molecule and/or the polypeptides, such as DAF binding or mimicking activity, etc. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules which encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules which encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acid molecules or polypeptides can be tested by routine experimentation for retention of structural relation or activity to the nucleic acids and/or polypeptides disclosed herein.

The invention also provides isolated fragments of nucleic acids encoding the antibodies described herein. The fragments can be used as probes in Southern blot assays to identify such nucleic acids, or can be used in amplification assays such as those employing PCR. Fragments also can be used to produce fusion proteins for generating antibodies or determining binding of the polypeptide fragments. Likewise, fragments can be employed to produce non-fused fragments of the polypeptides, useful, for example, in the preparation of anti-idiotype antibodies, assays, and the like.

A nucleic acid molecule, in one embodiment, is operably linked to a gene expression sequence which directs the expression of the nucleic acid molecule within a eukaryotic or prokaryotic cell. This can be for in vitro production of a polypeptide or for in vivo production of a polypeptide, including both manufacturing and therapeutic use. The “gene expression sequence” is any regulatory nucleotide sequence, such as a promoter sequence or promoter-enhancer combination, which facilitates the efficient transcription and translation of the nucleic acid molecule to which it is operably linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPTR), adenosine deaminase, pyruvate kinase, β-actin and other constitutive promoters. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of Moloney murine leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In general, the gene expression sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined nucleic acid. The gene expression sequences optionally includes enhancer sequences or upstream activator sequences as desired.

The nucleic acid sequence and the gene expression sequence are said to be “operably linked” when they are covalently linked in such a way as to place the transcription and/or translation of the coding sequence under the influence or control of the gene expression sequence. If it is desired that the sequence be translated into a functional protein, two DNA sequences are said to be operably linked if induction of a promoter in the 5′ gene expression sequence results in the transcription of the OE-1 sequence or the sequence of any of the peptides or fragments thereof, and if the nature of the linkage between the two DNA sequences does not (1) result in the introduction of a frame-shift mutation, (2) interfere with the ability of the promoter region to direct the transcription of the sequence, or (3) interfere with the ability of the corresponding RNA transcript to be translated into a protein. Thus, a gene expression sequence would be operably linked to a nucleic acid sequence if the gene expression sequence were capable of effecting transcription of that nucleic acid sequence such that the resulting transcript might be translated into the desired protein or polypeptide.

The nucleic acid molecules and the polypeptides of the invention can be delivered to the eukaryotic or prokaryotic cell alone or in association with a vector. In its broadest sense, a “vector” is any vehicle capable of facilitating: (1) delivery of a nucleic acid molecule or polypeptide to a target cell, (2) uptake of a nucleic acid or polypeptide by a target cell, or (3) expression of a nucleic acid molecule or polypeptide in a target cell. Vectors can also be used to facilitate delivery uptake on expression of inhibitors or inducers. Preferably, the vectors transport the nucleic acid or polypeptide into the target cell with reduced degradation relative to the extent of degradation that would result in the absence of the vector. Optionally, a “targeting ligand” can be attached to the vector to selectively deliver the vector to a cell which expresses on its surface the cognate receptor for the targeting ligand (e.g. a receptor, an antigen recognized by an antibody). In this manner, the vector (containing a nucleic acid or a polypeptide) can be selectively delivered to a specific cell. In general, the vectors useful in the invention are divided into two classes: biological vectors and chemical/physical vectors. Biological vectors are more useful for delivery and/or uptake of the nucleic acids described herein. Chemical/physical vectors are more useful for delivery and/or uptake of the nucleic acids or peptides discussed herein.

Biological vectors include, but are not limited to, plasmids, phagemids, viruses, other vehicles derived from viral or bacterial sources that have been manipulated by the insertion or incorporation of the nucleic acid sequences of the invention, and free nucleic acid fragments which can be linked to the nucleic acid sequences of the invention. Viral vectors are a preferred type of biological vector and include, but are not limited to, nucleic acid sequences from the following viruses: retroviruses, such as Moloney murine leukemia virus; Harvey murine sarcoma virus; murine mammary tumor virus; Rous sarcoma virus; adenoviruses; adeno-associated virus; SV40-type viruses; polyoma viruses; poxviruses; retroviruses; Epstein-Barr virus; papilloma viruses; herpes virus; vaccinia virus; and polio virus. One can readily employ other vectors not named but known in the art.

Preferred viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman C.O., New York, 1990, and Murry, E. J. Ed. “Methods in Molecular Biology,” vol. 7, Humana Press, Inc., Clifton, N.J., 1991.

Another preferred virus for certain applications is the adeno-associated virus, a double-stranded DNA virus. The adeno-associated virus can be engineered to be replication-deficient and is capable of infecting a wide range of cell types and species. It has further advantages, such as heat and lipid solvent stability, high transduction frequencies in cells of diverse lineages, and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extra chromosomal fashion.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA encoding a polypeptide or fragment or variant thereof. The heterologous DNA is placed under operable control of transcriptional elements to permit the expression of the heterologous DNA in the host cell.

Preferred systems for mRNA expression in mammalian cells are those such as pRc/CMV or pcDNA1 (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen, Carlsbad, Calif.), which contains an Epstein Barr virus (EBV) origin of replication, facilitating the maintenance of a plasmid as a multicopy extra chromosomal element. Another expression vector is the pEF-BOS plasmid containing the promoter of polypeptide Elongation Factor 1α, which stimulates efficiently transcription in vitro. The plasmid is described by Mishizuma and Nagata, 1990, Nuc. Acids Res. 18:5322, and its use in transfection experiments is disclosed by, for example, Demoulin, 1996, Mol. Cell. Biol. 16:4710-4716. Still another preferred expression vector is an adenovirus, described by Stratford-Perricaudet, 1992, J. Clin. Invest. 90:626-630, which is defective for E1 and E3 proteins.

In addition to the biological vectors, chemical/physical vectors may be used to deliver a nucleic acid molecule or polypeptide to a target cell and facilitate uptake thereof. As used herein, a “chemical/physical vector” refers to a natural or synthetic molecule, other than those derived from bacteriological or viral sources, capable of delivering the isolated nucleic acid molecule or polypeptide to a cell.

A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vesicles which are useful as delivery vectors in vivo or in vitro. It has been shown that large unilamellar vesicles (LUV), which range in size from 0.2-4.0μ can encapsulate large macromolecules. RNA, DNA, and intact virions can be encapsulated within the aqueous interior and be delivered to cells in a biologically active form (Fraley, et al., 1981, Trends Biochem. Sci., v. 6, p. 77). In order for a liposome to be an efficient nucleic acid transfer vector, one or more of the following characteristics should be present: (1) encapsulation of the nucleic acid of interest at high efficiency with retention of biological activity; (2) preferential and substantial binding to a target cell in comparison to non-target cells; (3) delivery of the aqueous contents of the vesicle to the target cell cytoplasm at high efficiency; and (4) accurate and effective expression of genetic information.

Liposomes may be targeted to a particular tissue by coupling the liposome to a specific ligand such as a monoclonal antibody, sugar, glycolipid, or protein. Ligands which may be useful for targeting a liposome to a particular cell will depend on the particular cell or tissue type. Additionally when the vector encapsulates a nucleic acid, the vector may be coupled to a nuclear targeting peptide, which will direct the nucleic acid molecules to the nucleus of the host cell.

Liposomes are commercially available from Gibco BRL, Carlsbad, Calif., for example, as LIPOFECTIN™ and LIPOFECTACE™, which are formed of cationic lipids such as N-[1-(2,3dioleyloxy)-propyl]-N,N,N-trimethylammonium chloride (DOTMA) and dimethyl dioctadecylammonium bromide (DDAB). Methods for making liposomes are well known in the art and have been described in many publications.

Other exemplary compositions that can be used to facilitate uptake by a target cell of the nucleic acid molecule include calcium phosphate and other chemical mediators of intracellular transport, microinjection compositions, electroporation and homologous recombination compositions (e.g., for integrating a nucleic acid molecule into a preselected location within a target cell chromosome).

The invention also embraces so-called expression kits, which allow the artisan to prepare a desired expression vector or vectors. Such expression kits include at least separate portions of the previously discussed coding sequences. Other components may be added, as desired, as long as the previously mentioned sequences, which are required, are included.

It will also be recognized that the invention embraces the use of the cDNA sequences in expression vectors to transfect host cells and cell lines, be these prokaryotic (e.g., E. coli), or eukaryotic (e.g., COS cells, yeast expression systems and recombinant baculovirus expression in insect cells). Especially useful are mammalian cells such as human, pig, goat, primate, dog, horse, cow etc. The cells may be of a wide variety of tissue types, and may be primary cells and cell lines. The expression vectors require that the pertinent sequence, i.e., those nucleic acids described herein, be operably linked to a promoter.

As discussed above, sequences encoding DAF (or portions of DAF) and anti-sense to DAF can be introduced into cells to upregulate or down regulate DAF expression, respectively. The nucleic acid sequence encoding DAF is reported by Caras et al., Nature, 1987, 325:545-549, the disclosure of which is incorporated herein by reference.

The invention also involves pharmaceutical preparations. When administered, the pharmaceutical preparations of the invention are applied in pharmaceutically acceptable amounts and in pharmaceutically acceptable compositions. Such preparations may routinely contain salts, buffering agents, preservatives, compatible carriers, and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrocholoric, hydrobromic, sulphuric, nitric, phosphoric, maleic, acetic, salicyclic, p-toluene sulfonic, tartaric, citric, methane sulfonic, formic, malonic, succinic, naphthalene-2-sulfonic, and benzene sulfonic. Also, pharmaceutically acceptable salts can be prepared as alkaline metal or alkaline earth salts, such as sodium, potassium or calcium salts.

Suitable buffering agents include: acetic acid and a salt (1-2% W/V); citric acid and a salt (1-3% W/V); boric acid and a salt (0.5-2.5% W/V); and phosphoric acid and a salt (0.8-2% W/V).

Suitable preservatives include benzalkonium chloride (0.003-0.03% W/V); chlorobutanol (0.3-0.9% W/V); parabens (0.01-0.25% W/V) and thimerosal (0.004-0.02% W/V).

The pharmaceutical preparations of the present invention contain a therapeutically effective amount of an agent of the invention included with a pharmaceutically-acceptable carrier. The term “pharmaceutically-acceptable carrier” as used herein means one or more compatible solid or liquid filler, dilutants or encapsulating substances which are suitable for administration to a human or other animal. The term “carrier” denotes an organic or inorganic ingredient, natural or synthetic, with which the active ingredient is combined to facilitate the application. The components of the pharmaceutical compositions are capable of being commingled with the molecules of the present invention, and with each other, in a manner such that there is no interaction which would substantially impair the desired pharmaceutical efficacy.

Compositions suitable for parenteral administration conveniently comprise a sterile preparation of the agents of the invention. This preparation may be formulated according to known methods, including but not limited to filtration, heat, radiation etc. The sterile preparation thus may be a sterile solution or suspension in a non-toxic parenterally-acceptable diluent or solvent. In addition, sterile, fixed oils are conventionally employed as a solvent or suspending medium. For this purpose any bland fixed oil may be employed including synthetic mono or di-glycerides. In addition, fatty acids such as oleic acid find use in the preparation of injectables. Carrier formulations suitable for oral, subcutaneous, intravenous, intramuscular, etc. can be found in Remington's Pharmaceutical Sciences, Mack Publishing Company, Easton, Pa., USA.

The agents of the invention are administered in effective amounts. An effective amount means that amount necessary to delay the onset of, inhibit the progression of, or halt altogether the onset or progression of the particular condition being treated, or manage the disease over a period of time. In general, an effective amount for treating an inflammatory condition will be that amount necessary to inhibit the onset or progression of inflammation or an amount administered chronically. This can be determined by observing symptoms of inflammatory conditions or by direct measurement of the extent of neutrophil transmigration. When administered to a subject, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation. It is preferred generally that a maximum dose be used, that is, the highest safe dose according to sound medical judgment.

Dosage may be adjusted appropriately to achieve desired drug levels, locally or systemically. Generally, daily oral doses of active compounds will be from about 0.01 mg/kg per day to 1000 mg/kg per day. It is expected that IV doses in the range of about 1 to 1000 mg/m.sup.2 per day will be effective. In the event that the response in a subject is insufficient at such doses, even higher doses (or effective higher doses by a different, more localized delivery route) may be employed to the extent that patient tolerance permits. Continuous IV dosing over, for example 24 hours or multiple doses per day are contemplated to achieve appropriate systemic levels of compounds.

A variety of administration routes are available. The particular mode selected will depend of course, upon the particular drug selected, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal, intradermal or parenteral routes. The term “parenteral” includes subcutaneous, intravenous, intramuscular, or infusion. Administration to mucosal surfaces is one important mode of administration, such as by oral administration, pulmonary administration, intestinal or colonic administration such as by enteric coating or enema, vaginal administration, suppository and the like.

The compositions may conveniently be presented in unit dosage form and may be prepared by any of the methods well known in the art of pharmacy. All methods include the step of bringing the conjugates of the invention into association with a carrier which constitutes one or more accessory ingredients. In general, the compositions are prepared by uniformly and intimately bringing the compounds into association with a liquid carrier, a finely divided solid carrier, or both, and then, if necessary, shaping the product.

Compositions suitable for oral administration may be presented as discrete units such as capsules, cachets, tablets, or lozenges, each containing a predetermined amount of the active compound. Other compositions include suspensions in aqueous liquors or non-aqueous liquid such as a syrup, an elixir, or an emulsion.

Other delivery systems can include time-release, delayed release or sustained release delivery systems. Such systems can avoid repeated administrations of the active compounds of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. In addition, a pump-based hardware delivery system can be used, some of which are adapted for implantation.

The compounds useful in the invention may be used alone, without other active agents. They also may be used together with other active agents, such as anti-inflammatory agents or agents known useful in treating the conditions described herein such as agents useful for treating inflammatory bowel disease. The agents may be delivered separately with other agents or in the form of a cocktail of two or more agents. A cocktail is a mixture of any one or more of the compounds useful with this invention with another active agent.

Useful anti-inflammatory agents include, but are not limited to, non-steroidal anti-inflammatory drugs such as Salicylic Acid, Acetylsalicylic Acid, Methyl Salicylate, Diflunisal, Salsalate, Olsalazine, Sulfasalazine, Acetaminophen, Indomethacin, Sulindac, Etodolac, Mefenamic Acid, Meclofenamate Sodium, Tolmetin, Ketorolac, Dichlofenac, Ibuprofen, Naproxen, Naproxen Sodium, Fenoprofen, Ketoprofen, Flurbinprofen, Oxaprozin, Piroxicam, Meloxicam, Ampiroxicam, Droxicam, Pivoxicam, Tenoxicam, Nabumetome, Phenylbutazone, Oxyphenbutazone, Antipyrine, Aminopyrine, Apazone and Nimesulide; leukotriene antagonists including, but not limited to, Zileuton, Aurothioglucose, Gold Sodium Thiomalate and Auranofin; and other anti-inflammatory agents including, but not limited to, Colchicine, Allopurinol, Probenecid, Sulfinpyrazone and Benzbromarone.

Other anti-inflammatory agents include Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

Anti-bacterial agents include: Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmenoxime Hydrochloride; Cefinetazole; Cefinetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofingin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine: Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate: Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafingin; Sisomicin; Sisomicin Sulfate; Spariloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin: Vancomycin; Vancomycin Hydrochloride; Virginiamycin; Zorbamycin.

Antifungal agents include: Acrisorcin; Ambruticin; Amphotericin B; Azaconazole; Azaserine; Basifungin; Bifonazole; Biphenamine Hydrochloride; Bispyrithione Magsulfex; Butoconazole Nitrate; Calcium Undecylenate; Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofungin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifingin Hydrochloride; Parconazole Hydrochloride; Partricin; Potassium Iodide; Proclonol; Pyrithione Zinc; Pyrrolnitrin; Rutamycin; Sanguinarium Chloride; Saperconazole: Scopafungin; Selenium Sulfide; Sinefingin; Sulconazole Nitrate; Terbinafine; Terconazole; Thiram; Ticlatone; Tioconazole; Tolciclate; Tolindate; Tolnaftate; Triacetin; Triafungin; Undecylenic Acid; Viridofulvin; Zinc Undecylenate; Zinoconazole Hydrochloride.

Anti-ulcerative agents include: Aceglutamide Aluminum; Cadexomer Iodine; Cetraxate Hydrochloride; Enisoprost; Isotiquimide; Lansoprazole; Lavoltidine Succinate; Misoprostol; Nizatidine; Nolinium Bromide; Pantoprazole; Pifamine; Pirenzepine Hydrochloride; Rabeprazole Sodium; Remiprostol; Roxatidine Acetate Hydrochloride; Sucralfate; Sucrosofate Potassium; Tolimidone.

This invention is not limited in its application to the details of construction and the arrangement of components set forth herein or illustrated in the drawings. The invention is capable of other embodiments and of being practiced or of being carried out in various ways. Also, the phraseology and terminology used herein is for the purpose of description and should not be regarded as limiting. Those skilled in the art will be able to recognize with no more than routine experimentation numerous equivalents to the specific products and processes described above. Such equivalents are intended to be included within the scope of the appended claims.

### EXAMPLES

### Example 1

### Fusion Screen and Functional Characterization of mAb OE-1

The purpose of this example was to identify other apical epithelial ligands important in final stages of PMN transmigration. To do this, a panel of monoclonal antibodies generated to epithelial plasma membrane antigen was screened. This screen identified one monoclonal antibody (mAb), termed OE-1, which recognized an apical epitope and inhibited physiologically-directed PMN transmigration. Extensions of these observations identified the OE-1 antigen as decay accelerating factor (DAF), a GPI-link ˜80 kDa protein originally described as an inhibitor of autologous complement lysis.

Materials and Methods

Cell Culture. T84 cells, Caco2 cells and KB cells were grown on permeable 0.33 cm² ring-supported polycarbonate filters (0.4 μm pore size) (Costar Corp., Cambridge, Mass.) or plastic polystyrene tissue culture dishes (Costar Corp., Cambridge, Mass.), as indicated, using previously described techniques (Turner et al., 1997, Am. J. Physiol. 273: C1378-85; Madianos et al., 1996, Infect. Immun. 64: 660-4; Dharmsathaphorn, et al., 1990, Methods Enzymol. 192: 354-89). To plate inverted inserts, transwell inserts were placed upside in a large Petri dish and 80 μl of cell suspension was plated on each insert. The inserts were incubated overnight at 37° C. in a humidified incubator with 5% CO₂. The following day the inserts were flipped into DMEM supplemented with 20% FBS. Where indicated, human microvascular endothelial cells were cultured as previously described (Collard, et al., 2002, J. Biol. Chem., 2002: 14801-14811). The non-transformed human oral keratinocyte cell line OKF6 were grown as previously described (Dickson, et al., 2000, Mol. Cell. Biol. 20: 1436-47).

Monoclonal antibody production. KB cell plasma membranes were used as an antigen to generate a panel of antibodies for functional screening. Plasma membranes were isolated using nitrogen cavitation (200 psi, 8 min., 4° C.) as previously described (Colgan et al., 1993, J. Cell. Biol. 120: 785-795). Mice were injected intraperitonally with a 50:50 mix of Titermax (Titermax USA, Inc., Norcross, Ga.) and KB membrane protein (50 μg). Mice were inoculated every two weeks for 6 weeks with 25 μg KB membranes in sterile PBS. Splenocytes were harvested, washed in PBS and mixed 2:1 with hybridoma cells in the presence of 50% polyethylene glycol. Cells were plated in DMEM supplemented with 1% HAT for selection. Single clones were obtained by limiting dilution (number of clones=920). Cultures of KB cells in 96-well plates were used in a ecto-ELISA to select for clones producing antibodies to epithelial surface proteins (number of clones=510). Supernatants from these clones were used PMN transmigration assays to select for antibodies which inhibited transmigration by 50%. Of these, one antibody (subclone OE-1, IgG2a) was further characterized.

Human neutrophil isolation. PMN were freshly isolated from whole blood obtained by venipuncture from healthy human donors and anticoagulated with acid citrate dextrose as described previously (Henson and Oades, 1975, J. Clin. Invest. 56: 1053-61). Briefly, plasma and mononuclear cells were removed by aspiration from the buffy coat following centrifugation (400×g, 20 min) at room temperature. Red blood cells were sedimented using 2% gelatin, and residual RBC were removed by lysis in ice cold NH₄Cl buffer. PMN were >90% pure as determined by microscopic evaluation. PMN were resuspended to a final concentration of 5×10⁷ in Hank's balanced salts solution (HBSS; with 10 mM Hepes; pH 7.4; and without Ca²⁺ or Mg²⁺; Sigma, St. Louis. Mo.). PMN were used within 2 hours of isolation.

Transmigration assay. PMN transmigration assays were performed as previously described (Nicholson-Weller et al., 1994, J. Lab. Clin. Med. 123: 485-91). All epithelial experiments were performed in the physiologically-relevant basolateral-to-apical direction (i.e. inverted monolayers), unless otherwise indicated, and all HMVEC transmigration studies were performed in the apical-to-basolateral direction. Briefly, PMN (1×10⁶) were added to the upper chambers of transwell inserts in which T84, KB or Caco2 cell monolayers, as indicated, were plated on the opposing side. A chemotactic gradient was established by adding n-formyl-methionyl-leucyl-phenylalanine (fMLP) to the lower chambers (1 μM for Caco2 and T84 epithelia and 10 nM for KB cells and HMVEC). PMN transmigration was carried out at 37° C. for 1 hour with KB and HMVEC monolayers and 2 hrs for T84 monolayers. Transmigrated PMN were quantified by assaying for the PMN azurophilic marker myeloperoxidase (MPO), as previously described (Lisanti et al., 1990, J. Memb. Biol. 113: 155-167). Briefly, transmigrated PMN were lysed by the addition of Triton X-100 to a final concentration 0.5%. The samples were acidified with citrate buffer (final concentration 100 mM; pH 4.2). An aliquot of sample (70 μl) was added to an equal volume of ABTS solution (1 mM ABTS (2.2′-Azino-bis(3-ethylbenzo-thiazoline-6-sulfonic acid), 0.03% H₂O₂, 100 mM sodium citrate buffer; pH 4.2) in a 96-well plate. The resulting color was quantitated on a plate reader at 405 nm. For kinetic transmigration and post-transmigration adhesion assays, monolayers were removed at the desired time point and placed in a new 24-well plate with HBSS and spun at 50×g for 5 min to dislodge PMN adherent to the monolayer (Liu et al., 2001, J. Biol. Chem. 276: 40156-66). PMN were quantified by MPO assay as stated above. Where indicated, polyclonal DAF antisera (a kind gift from Dr. B. Paul Morgan, University of Wales) or control polyclonal platelet-endothelial cell adhesion (PE-CAM, a kind gift from Dr. Joyce Bischoff, Children's Hospital and Harvard Medical School) antisera were used to assess transmigration.

Data Analysis. PMN transmigration and ELISA data were compared by two-factor analysis of variance (ANOVA) or by Student's t-Test, where appropriate. Values are expressed as the mean and S.E.M. from at least 3 separate experiments.

Results

Fusion results. One fusion of splenocytes from mice immunized with KB cell membranes yielded 920 antibody producing clones. Each clone was screened for reactivity to intact KB cells and for inhibition of PMN transmigration across KB cell monolayers. In total, 510 clones were epithelial reactive and of these, 31 clones significantly influenced PMN transmigration (n=29 inhibited PMN migration >50%, n=2 promoted PMN migration >50%). Of these, one subclone (IgG2a, termed clone OE-1) which inhibited PMN migration in this screen also bound dominantly to the apical cell surface (see later), and as such, was further characterized as an apical epithelial ligand for PMN.

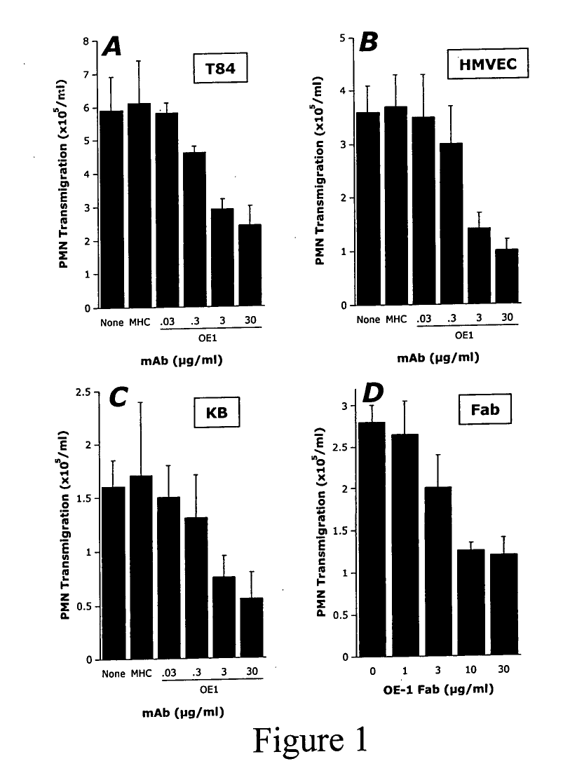

Functional characterization of mAb OE-1. OE-1 was tested on two epithelial cell lines (KB cells and T84 cells) and one non-transformed primary culture of microvascular endothelial cells (HMVEC) to determine the influence on PMN transmigration. As shown in FIG. 1, OE-1 significantly inhibited PMN migration in a concentration-dependent manner (for each, p<0.025 by ANOVA), with 3 μg/ml inhibiting PMN transmigration by ≧50% compared to either no mAb (p<0.001) or to our binding control W6/32 directed against MHC class I (p<0.01, see FIG. 1A-C). PMN migration across T84 monolayers (panel A, basolateral-to-apical direction), KB monolayers (panel B, basolateral-to-apical direction) or HMVEC monolayers (panel C, apical-to-basolateral direction) in response to a fMLP gradient was carried out in the presence or absence of indicated concentrations of intact OE-1, control antibody (W6/32) or Fab fragments (panel D, KB cell transmigration, basolateral-to-apical direction). Data are presented as mean±s.e.m. (n=3).

Recent studies have suggested that PMN binding to epithelial bound antibody via surface Fc receptors may influence PMN trafficking through epithelia (Reaves et al., 2001, Am. J. Physiol. Gastrointest. Liver Physiol. 280: G746-54). To rule out the involvement of PMN Fc receptor ligation in this activity, we enzymatically cleaved OE-1 with papain to generate Fab fragments, and tested such Fab fragments for inhibition of PMN migration. As shown in FIG. 1D, Fab fragments of OE-1 also inhibited PMN transmigration (p<0.01), indicating that such findings are not likely related to Fc-mediated interactions with PMN.

Previous studies have indicated that PMN transmigration across polarized epithelia can vary dependent on the direction of migration (Parkos et al., 1991, J. Clin. Invest. 88: 1605-12; Colgan et al., 1993, J. Cell. Biol. 120: 785-795). As such, it was examined whether OE-1 influenced PMN transmigration in a polarized fashion. Interestingly, inhibition of transmigration by OE-1 was independent of the direction of migration across electrically confluent (TER>1000 ohm·cm²), polarized T84 epithelia. Relative to mAb control W6/32 (10 μg/ml), OE-1 (10 μg/ml) inhibited PMN migration by 69±8% in the non-physiologic apical-to-basolateral direction (p<0.01), and by 73±10% in the physiologically relevant basolateral-to-apical direction (p<0.01). In total, these results suggest that the OE-1 antigen represents a surface protein important to successful PMN transmigration across both endothelial and epithelial cell monolayers.

### Example 2

### Biochemical Characterization of OE-1 Antigen

Materials and Methods

Cell culture, human neutrophil isolation, mAb production, transmigration assays and data analysis materials and methods were used as described in Example 1.

Tryptic digestion and identification of OE-1 antigen. Bulk Ag was purified from ˜500 cm² of confluent KB plasma membranes using OE-1 coupled affinity column (CnBr activated sepharose 4B, Pierce, Rockville, Ill.) as described previously (Parkos, et al., 1996, J. Cell Biol. 132: 437-450). Antigen was eluted at low pH (150 mM NaCl, 100 mM glycine/HCl, pH 2.5 containing 1% n-octylglucoside 1%). The resulting eluant was pH neutralized, resolved by SDS PAGE and bands were localized by Coomassie stain. The resulting ˜80 kDa band (˜100 pmol) was extracted and submitted to the Dana Farber Cancer Institute Peptide Core Facility (Boston, Mass.) for trypsin digestion and microsequence analysis.

Immunofluorescent staining of epithelial monolayers. Caco2 cells were grown to confluency on membrane permeable filters. Following transmigration, the inserts were fixed for 10 minutes at RT in 1% paraformaldehyde in cacodylate buffer (0.1M sodium cacodylate; pH 7.4, 0.72% sucrose). After washing once with PBS, the cells were stained for 1 hour at room temperature with a monoclonal OE-1 (130 μg/ml), polyclonal anti-DAF (1:100) or monoclonal anti-CD11b (60 μg/ml; clone 44a). After washing twice in PBS, the monolayers were incubated with either goat anti-mouse Oregon Green (1 μg/ml) or goat anti-rabbit Texas Red (1 μg/ml). Fluorescent secondary antibodies were purchased from Molecular Probes (Eugene, Oreg.). Stained inserts were carefully excised and mounted in polyvinyl alcohol mounting media. BioRad Laser Sharp imaging software (BioRad, Hercules, Calif.) was used for confocal imaging and processing.

Immunoblotting experiments. Indicated cells were grown to confluency on 100 mm plastic Petri dishes. The monolayers were lysed for 10 min in 1 ml lysis buffer (150 mM NaCl, 25 mM Tris, pH 8.0, 5 mM EDTA, 2% n-octylglucoside, and 10% mammalian tissue protease inhibitor cocktail; Sigma, St. Louis, Mo.), scraped and collected into microfuge tubes. After spinning at 14,000×g to remove cell debris, the pellet was discarded. Proteins were solublized in non-reducing Laemmli sample buffer and heated to 100° C. for 5 minutes. Samples were resolved on a 10% polyacrylamide gel and transferred to nitrocellulose membranes. The membranes were blocked 1 hr at room temperature in PBS supplemented with 0.2% Tween-20 (PBS-T) and 4% BSA. The membranes were incubated in 3 μg/ml OE-1 in PBS-T for 1 hr at room temp, followed by 10 minute washes in PBS-T. The membranes were then incubated in 1:10,000 goat-anti mouse IgG (ICN/Cappel, Cosa Mesa Calif.), conjugated to horse radish-peroxidase for 1 hr at room temperature. The wash was repeated and proteins were detected by enhanced chemiluminescence.

Sequential immunoprecipitations. Cells were grown to confluency on 100 mm plastic Petri dishes. The monolayers were lysed with 1 ml lysis buffer. Cellular debris was removed by centrifugation and the lysates were pre-cleared with 25 μl of a 50% protein G-sepharose slurry (Amersham Pharmacia, Piscataway, N.J.) for 2 hours at 4° C. OE-1 (20 μg) or 20 μg polyclonal anti-DAF was added to 1 ml of lysate, rotated overnight at 4° C., then subjected to capture with 50 μl of 50% protein G-sepharose slurry. After the protein G-sepharose beads had been removed, the immunoprecipitation reaction was repeated two more times to effectively remove the OE-1 antigen. Finally, OE-1 and anti-DAF immunoprecipitated lysates were subjected to a final immunoprecipitation reaction with anti-DAF or OE-1 antibody, respectively. The captured antigen from each immunoprecipitation reaction was washed three times in immunoprecipitation wash buffer. After solubilization in Laemmli sample buffer, protein were resolved by SDS PAGE and visualized by western blotting with OE-1.

Differential biotinylation of apical and basolateral surface proteins. T84 cells were grown to confluence on 0.5 μm polycarbonate transwell inserts. The monolayers were washed once in HBSS. Sulfo-NHS-Biotin (Pierce, Rockford, Ill.) was diluted in HBSS to a final concentration of 1 mM and added apically or basolaterally for 10 minutes at 4° C. The monolayers were washed repeatedly with HBSS containing 150 mM NH₄CL to quench the residual biotin. Monolayers were lysed in lysis buffer for 10 minutes at 4° C. After removal of cellular debris by centrifugation, 20 μg OE-1 was added to immunoprecipitate DAF. Biotinylated cell surface DAF was detected with avidin-horseradish peroxidase (Pierce, Rockford, Ill.).

Results

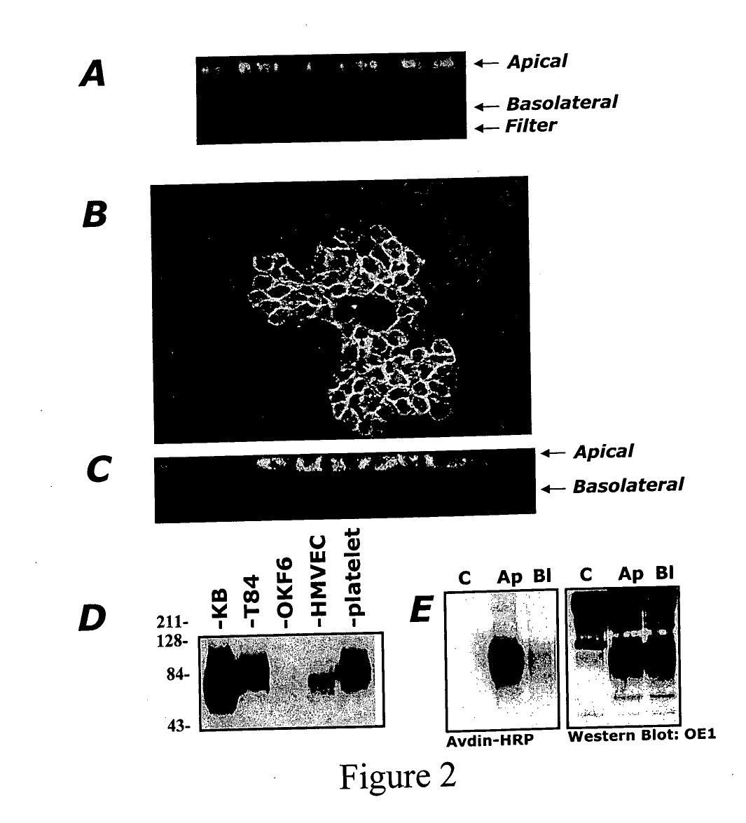

Biochemical characterization of the OE-1 antigen. The relative nature and localization of OE-1 antigen was determined as shown in FIG. 2. In panel A, confocal immunofluorescence was used to define the pattern of OE-1 antigen expression. Shown here is a x-z orientation series demonstrating nearly complete localization to the apical and subapical membrane compartment. In panel B, is an apical confocal section localizing OE-1 with the PMN marker CD11b/18 (clone 44a) following PMN transmigration. Panel C represents a x-z orientation series of panel B. In panel D, equal amounts of lysates from indicated cell types were resolved on 10% SDS-PAGE and immunoblotted with OE-1. The OE-1 antigen is expressed in varying amounts in the different cell types. The differences in molecular weights are likely due to differential glycosylation. Epithelial cell lines (KB, T84) express the highest levels of OE-1 antigen, followed by cells normally in close contact with complement proteins (HMVEC, platelets). Primary cell lines such as OKF6 (keratinocytes) express lesser amounts of the OE-1 antigen. In panel E, confluent T84 monolayers were biotinylated on the apical (Ap) or basolateral (Bl) surface. Lysates were immunoprecipitated with either an isotype control antibody (C) or OE-1. Proteins were visualized with avidin-HRP to identify only surface, biotinylated DAF (left) or with OE-1 to demonstrate total DAF (right). Lanes labeled C represent isotype immunoprecipitation control samples.

Confocal immunolocalization was utilized to determine the overall localization on T84 epithelia. As shown in FIG. 2A, the OE-1 epitope localized exclusively to the apical membrane surface and to subapical membrane domains of polarized cells grown on membrane permeable supports.

Localization of the antigen following PMN transmigration in the presence of OE-1 revealed that the OE-1 epitope remains localized to the apical domain and that PMN (localized with anti-CD11b mAb 44a) appear to “cluster” in distinct regions on the apical surface in the presence of OE-1 (see FIGS. 2B and 2C). This clustering of PMN is consistent with previous observations (Colgan et al., 1993, J. Cell. Biol. 120: 785-795), and is likely a result of localized epithelial barrier disruptions, with resultant increases in flux of chemoattractant.

Western blot analysis of the OE-1 epitope revealed a heavily glycosylated 70-80 kDa protein expressed to varying degrees on a number of different cell types, including epithelial cells, endothelial cells and platelets (se FIG. 2D). The relatively small differences in apparent molecular weight between different cell lines likely represent cell tissue-specific differences in glycosylation (Nicholson-Weller et al., 1994, J. Lab. Clin. Med. 123: 485-91). This antibody did not Western blot under reducing conditions (data not shown). Immunoprecipitation of differential surface biotinylated T84 cells confirmed our confocal localization results above, with nearly exclusive expression demonstrated in cells labeled from the apical, but not basolateral, surface (FIG. 2E). Such results suggest that the OE-1 antigen represents a heavily glycosylated, ˜70-80 kDa protein expressed on the apical membrane surface.

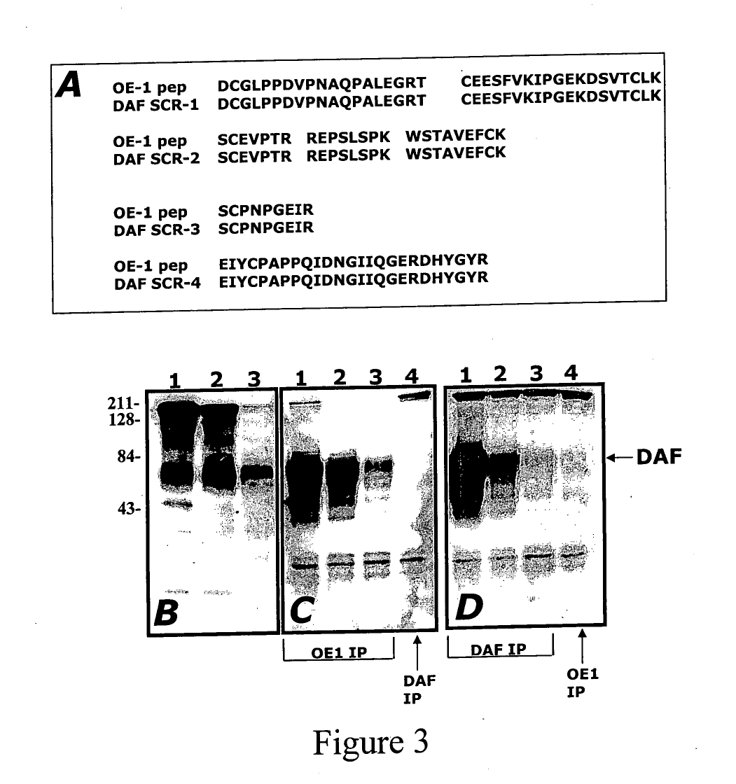

Identification of the OE-1 antigen as DAF. Experiments were next performed to identify the antigen recognized by OE-1 and the results are shown in FIG. 3. Panel A demonstrates tryptic peptides derived from affinity purified OE-1 antigen (OE-1 pep) aligned with DAF SCR domains 1-4. To confirm the identity of the OE-1 antigen as human CD55, IP reactions were performed. In panel B, T84 lysates were immunoprecipitated with OE-1 (lane 1) or a polyclonal anti-DAF (lane 2). The resolved proteins were immunoblotted using OE-1 under non-reducing conditions. Notice both antibodies immunoprecipitated identical proteins. Lane 3 is cell lysate run as a marker for DAF. In panel C, a single lysate was sequentially immunoprecipitated 3 times using 5 μl of anti-DAF per reaction. The captured protein from each IP reaction is shown in lanes 1-3. Note diminishing amounts of DAF recovered in each reaction. After the third reaction, the lysate was immunoprecipitated with 10 μg of OE-1 (lane 4). Lack of a recoverable protein confirms the identity of the OE-1 antigen as CD55. Panel D is identical to the experiment in panel C, except the sequential IPs were first done using OE-1 and the final IP was done using polyclonal anti-DAF.

Approximately 500 cm² of confluent KB cells were utilized to obtain sufficient quantities of OE-1 antigen for microsequence analysis. The antigen was digested with trypsin and seven tryptic peptides resulting from mass spectroscopy showed direct sequence homology (FIG. 3A) with CD55, also termed decay-accelerating factor (DAF), a glycoprotein organized into four homologous short consensus repeats (SCR, also called complement control protein repeats) classically viewed as an inhibitor of autologous complement lysis. In support of this identification, and as suggested by our localization and western blotting experiments (FIG. 2), DAF is a heavily glycosylated ˜70-80 kDa protein widely expressed in many cells and tissue types. In addition, on most cell types DAF exists predominantly as a GPI-anchored protein (Nicholson-Weller et al., 1994, J. Lab. Clin. Med. 123: 485-91). In support of our apical localization results (FIG. 2), GPI-anchored proteins are predominantly expressed only on the apical membrane in polarized epithelial cells (Lisanti et al., 1990, J. Memb. Biol. 113: 155-167).

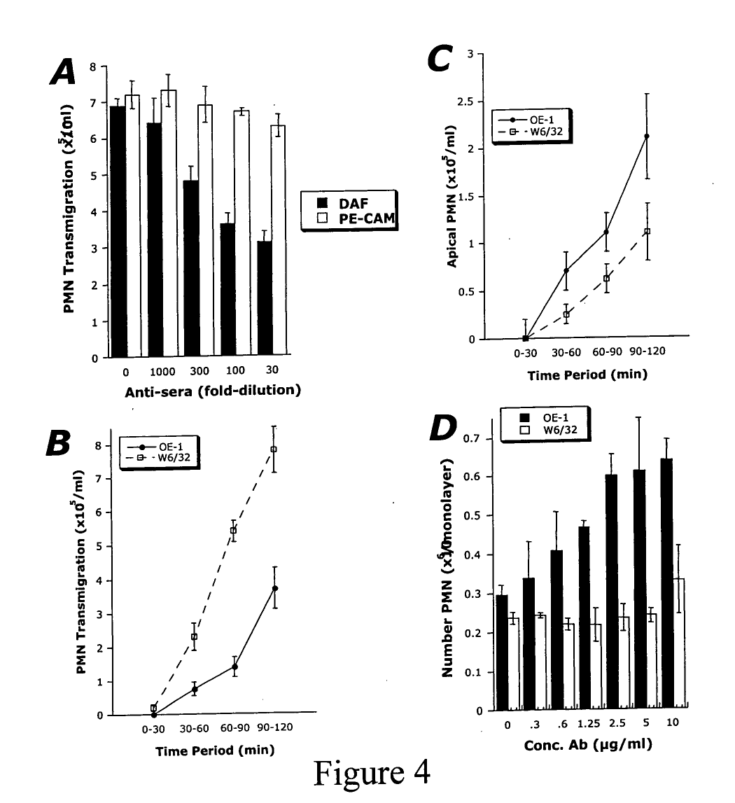

To further confirm the identity of DAF as the OE-1 antigen, immunoprecipitation and immunodepletion experiments were performed. As shown in FIG. 3B, T84 lysates were immunoprecipitated with either OE-1 (lane 1), or polyclonal anti-DAF (lane 2). Lane 3 is platelet lysate as a standard. As can be seen, both immunoprecipitation reactions pull down proteins of a similar molecular mass and degree of glycosylation. For the immunodepletion experiments, a single lysate was repeatedly immunoprecipitated with OE-1 (FIG. 3C), or polyclonal anti-DAF (FIG. 3D). Each lysate was immunoprecipitated 3 times to deplete the antibody binding protein, followed by a single immunoprecipitation with the opposing antibody (polyclonal anti-DAF or OE-1, respectively). As can be seen, the nearly complete absence of signal in the cross immunoprecipitation reactions (lane 4 of FIGS. 3C and 3D) confirmed that OE-1 and anti-DAF bind to identical antigens from epithelial lysates. Similarly, as shown in FIG. 4A, polyclonal antisera against DAF, but not control antisera to PE-CAM, inhibited PMN transmigration in a concentration-dependent manner (p<0.025 by ANOVA). Taken together, these studies identify the OE-1 antigen as DAF and indicate that DAF is an apically localized epithelial protein which functions, at least in part, to modulate PMN migration.

### Example 3

### Apical DAF Represents an Anti-Adhesive PMN Ligand

Materials and Methods

Cell culture, human PMN isolation, mAb production, transmigration assays, data analysis, tryptic digestion, immunofluorescent staining, immunoblotting, immunoprecipitations and biotinylation materials and methods were used as described in the previous examples.

PMN adhesion assay. PMN adhesion to confluent T84 epithelial cells was performed using modifications of a previous protocol (Zund et al., 1997, Am. J. Physiol. (Cell) 273: C 1571-C1580). Briefly, for studies of adhesion, human PMN were labelled for 30 min. at 37° C. with 2′7′-bis(carboxyethyl)-5 (6)-carboxyfluorescein pentaacetoxymethyl ester (BCECF-AM, 5 μM final concentration Calbiochem, San Diego, Calif.) and washed three times in HBSS. Epithelial monolayers grown on 24 well plates were pre-incubated with mAb OE-1 or control W6/32 at indicated concentrations for 10 min at 37° C. BCECF labelled PMN (2×10⁶/monolayer) were added to washed epithelial monolayers containing 1 OriM FMLP, plates were centrifuged at 150×g for 4 min to uniformly settle PMN, and adhesion was allowed for 10 min at 37° C. Monolayers were gently washed three times with HBSS and fluorescence intensity (excitation, 485 nm; emission, 530 nm) was measured on a fluorescent plate reader (Cytofluor™ 2300, Millipore Inc., Bedford, Mass.). Adherent cell numbers were determined from standard curves generated by serial dilution of known PMN numbers diluted in HBSS. All data were normalized for background fluorescence by subtraction of fluorescence intensity of samples collected from monolayers incubated in buffer only, without addition of PMN.

DAF suppression studies. Caco2 cells were chosen for these experiments because they are more easily transfected than T84 cells. Confluent Caco2 monolayers were dislodged from T175 flasks with trypsin. Cells were washed once in serum-free DMEM. To 100 μl serum free media, 10 μl DAF siRNA ribonucleotide (20 μM each of sense 5′-AAUUCCUGGCGAGAAGGACUCdTdT-3′ (SEQ ID NO: 31) and antisense 3′-dTdTUUAAGGACCGCUCUUCCUGAG-5′ (SEQ ID NO: 32), synthesized by Xeragon, Inc., Germantown, Md.) or 10 μl phosphorothioate derivatives of DAF antisense oligonucleotide (20 μM; 5′-CGTGTCTCAGAGACCGACTT-3′ (SEQ ID NO: 33), synthesized by Oligo's Etc., Wilsonville, Oreg.) was added. In both cases, a control oligonucleotide (5′-ATG GAG GGC GCC GGC-3′, SEQ ID NO: 34) was used in parallel. To these solutions 10 μl Superfect transfection reagent (Qiagen, Valencia, Calif.) was added. The tubes were vortexed and incubated at room temperature for 10 min to allow transfection complexes formation. Following this incubation, 400 μl of serum containing media was added to the complexes, followed by 800 μl of cell suspension. The cells and complexes were mixed well and 80 μl of cell suspension was plated on the underside of each transwell insert. The inserts were incubated upside down overnight at 37° C. in a humidified incubator containing 5% CO₂. The following day the inserts were flipped into DMEM containing 20% FBS. Monolayers were used after 4 days of incubation.

Results

Apical DAF represents an anti-adhesive PMN ligand. The functional role of epithelial DAF in modulation of PMN transmigration was determined. It was reasoned that if DAF is functional from the apical membrane surface of epithelia, it may represent a terminal step in PMN migration, and as such, may contribute significantly to the kinetics of PMN accumulation at the apical membrane domain. Three approaches were used to define these principles. First, the influence of OE-1 on the kinetics of PMN transmigration was examined. To do this, a previously described approach which quantitates PMN migration from the same set of monolayers over blocks of time (Reaves et al., 2001, Am. J. Physiol. Gastrointest. Liver Physiol. 280: G746-54; Liu et al., 2001, J. Biol. Chem. 276: 40156-66) was utilized. In these experiments, PMN were assessed every 30 min over a 2 hr period. The results are shown in FIG. 4. Panel A shows the influence of DAF antisera on PMN transepithelial migration. Epithelia were exposed to indicated concentrations of DAF (black bars) or control PE-CAM (clear bars) antisera and assessed for PMN transmigration. In panels B and C, the influence of OE-1 (dashed line) or control W6/32 (solid line) on the kinetics of transmigration was assessed. Panel B represents the number of PMN which completely traverse the epithelial monolayer, and panel C represents the number of PMN which remain attached to the apical epithelial membrane. Data are presented as mean±s.e.m. (n=4). In panel D, the influence of indicated concentrations of mAb OE-1 or control mAb W6/32 on FMLP-stimulated PMN adhesion to confluent T84 cells was assessed. Data are presented as mean±s.e.m. (n=4).

As shown in FIG. 4B, compared to control W6/32, the presence of OE-1 significantly slowed PMN transmigration across T84 epithelial monolayers (p<0.01 by ANOVA). Significant differences were observed as early as 30-60 minutes, and were evident as late as 150 minutes (data not shown). More importantly, this decrease in PMN transmigration shown in FIG. 4B was near completely explained by accumulation of PMN on the apical surface of epithelia (FIG. 4C). Indeed, as early as 30-60 min, the number of apical PMN significantly increased in the presence of OE-1 (p<0.05 compared to W6/32) and remained elevated through the 120 min period. Such findings indicate that rather than inhibiting PMN transmigration, OE-1 promotes the apical accumulation of PMN on epithelia.

Second, it was reasoned that if DAF functions as anti-adhesive molecule, then OE-1 should promote adhesion to the apical surface of epithelia. To test this idea, epithelial-PMN adhesion assays were performed. As shown in FIG. 4D, pre-incubation of epithelia with increasing concentrations of OE-1 and determination of FMLP-stimulated PMN adhesion revealed that OE-1 increases PMN adhesion in a concentration-dependent fashion (p<0.025 by ANOVA). Such findings were not related to Fc receptor mediated adhesion, since isotype control antibodies (clone W6/32) resulted in no significant increase in adhesion (p=not significant).

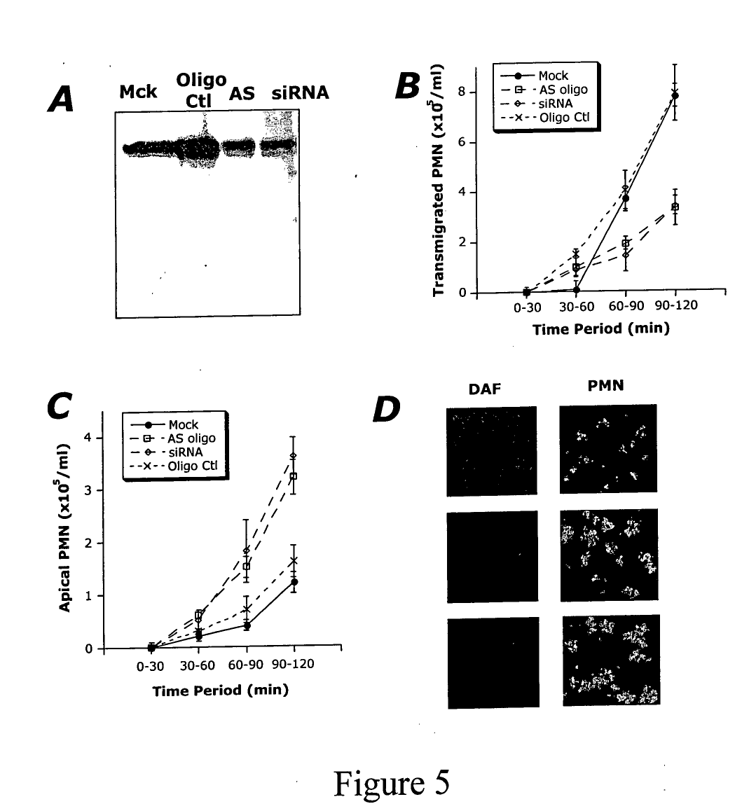

As a third approach, antisense oligonucleotides and siRNA technologies were utilized to diminish surface expressed DAF and examined the kinetics of PMN migration across such monolayers of epithelia. The results of this experiment are shown in FIG. 5. Panel A shows a representative Western blot analysis of total cellular DAF protein in Caco2 cells exposed to mock conditions (transfection loading reagent only), control oligonucleotide, DAF antisense oligonucleotide or DAF siRNA. In panels B and C, the influence of siRNA (diamonds), antisense oligonucleotide (squares) on the kinetics of transmigration was assessed. Panel B represents the number of PMN which completely traverse the epithelial monolayer and panel C represents the number of PMN which remain attached to the apical epithelial membrane. Also shown are mock conditions (closed circles) and oligonucleotide control conditions (crosses). Data are presented as mean±s.e.m. (n=4). Panel D shows immunofluorescence photomicrographs of PMN at the epithelial apical surface after transmigration in monolayers loaded with siRNA, antisense oligonucleotide (AS) or mock treatment. Decreased DAF expression is associated with increased PMN clustering on the apical surface of epithelia (compare density of clusters in mock vs. siRNA/AS conditions).

As determined by immunoprecipitation of surface biotinylated protein (FIG. 5A), antisense oligonucleotides and siRNA decreased surface expressed DAF by 68±10% and 63±12% relative to oligonucleotide controls (n=3 determinations). Using cells treated in this manner, we assessed transmigration and apical PMN accumulation. As shown in FIG. 5B, PMN transmigration across both antisense and siRNA exposed cells was significantly diminished relative to either mock treated or oligonucleotide control treated monolayers (p<0.025 for both by ANOVA). Diminished PMN transmigration in DAF-depleted cells was less obvious when measured at early time points, and became most apparent beyond 90 min. Conversely, as shown in FIG. 5C, assessment of apical PMN in DAF-depleted epithelia revealed a significant increase over time in a manner similar to mAb OE-1 exposure (p<0.05 for both by ANOVA). In addition, we determined whether is was possible to distinguish such biochemical differences at the morphological level. As shown in FIG. 5D, confocal localization of PMN in mock and antisense/siRNA treated monolayers following transmigration revealed increased PMN represented as large aggregations of PMN particularly prominent on the apical surface of DAF-depleted epithelia. Taken together, these results support our findings with mAb OE-1 and suggest that DAF represents an apically localized, anti-adhesive PMN ligand which significantly influences the kinetics of PMN trafficking through epithelial monolayers.

### Example 4

### Phage Display Mapping of the OE-1 Binding Epitope

Materials and Methods

Cell culture, human PMN isolation, mAb production, transmigration assays, data analysis, tryptic digestion, immunofluorescent staining, immunoblotting, immunoprecipitations and biotinylation materials and methods were used as described in the previous examples.

Phage panning of OE-1. The OE-1 binding epitope was analyzed using phage display (Burritt et al., 1998, J. Biol. Chem. 273:24847-52). Briefly, sepharose-coupled OE-1 was incubated with 20-μL aliquots of the random 9-mer LL9 phage library (˜5×10¹⁰ phage) in 1 mL HBSS containing 0.1% bovine serum albumin (phage buffer) for 2 hours at 4° C. or for 1 hour at 20° C. in a 1.5-ml microcentrifuge tube as described previously (Mazzucchelli et al., 1999, Blood 93: 1738-48). Antibody complexes were then washed five times with phage buffer and bound phage were eluted for 5 minutes in 2 mL of 0.1M glycine (pH 2.2) followed by addition of phage buffer containing 0.5% Tween 20 to the remaining cell pellet. After neutralization with Tris buffer pH 8.1, the titer of phage was determined in each fraction by plaque assay according to standard procedures. Phage elutes were then amplified in “starved” K91 E. coli on solid LB agar containing 75 μg/mL kanamycin, purified by precipitation with polyethylene glycol, and resuspended in 600 μL NaCl/HEPES buffer. An aliquot (20 μL) of purified phage was subsequently re-applied to a new aliquot of sepharose-coupled OE-1 for a total of three affinity purification and two amplification steps. Individual phage clones were then isolated, amplified, and the random peptide sequences were deduced after sequencing the unique nucleotide region of the pIII protein. As a control for OE-1-specific selection, parallel phage affinity purification steps were carried out in a 1.5-mL microcentrifuge tube in the absence of OE-1, and a sample of the recovered phage was analyzed by sequencing of the DNA insert. Consensus sequence peptide (amino acid sequence EVEHWYRSG, SEQ ID NO: 36), as well as scrambled control peptide (amino acid sequence SPLAQAVRSSSR, SEQ ID NO: 35) was commercially synthesized (BioWorld Inc., Dublin, Ohio) and tested in transmigration and OE-1 bind assays as described above.

In subsets of experiments, peptides were biotinylated (sulfo-NHS-Biotin, Pierce, Rockford, Ill.), purified by reversed phase HPLC, and tested for binding to PMN. Briefly, PMN (10⁷ PMN in 1 ml) were incubated with biotinylated peptide DAF peptide or control peptide (each at 10 μg/ml) at 4° C. for 1 hr. PMN were washed three times in HBSS, and incubated with rhodamine-labeled steptavidin (Roche Diagnostics, Indianapolis, Ind.). Samples were aliquoted on a 96 well plate and fluorescence intensity (excitation, 550 nm; emission, 580 nm), expressed as relative fluorescence units (RFU) was measured on a fluorescent plate reader (Cytofluor™2300, Millipore Inc., Bedford, Mass.).

Results

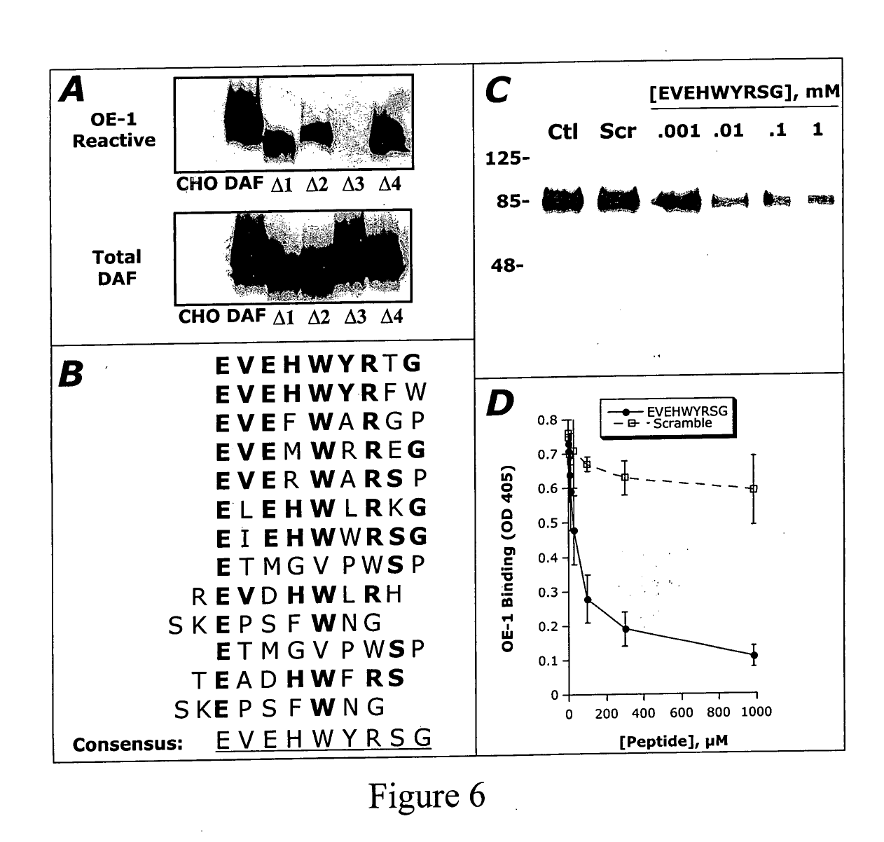

Phage display mapping of the OE-1 binding epitope. Initially, OE-1-based western blotting was used to screen CHO transfectants expressing wild-type human DAF and truncations of DAF SCR domains 1-4, as described previously (Coyne et al., 1992, J. Immunol. 149: 2906-13). The results of this study are shown in FIG. 6. In panel A, lysates from CHO cell mutants expressing DAF with individual SCR domains deleted were used to map which SCR domain contained the OE-1 epitope. Lysates were resolved by SDS-PAGE and immunoblotted with OE-1 (upper panel) or polyclonal DAF antisera (lower panel) to assess total DAF. Lanes: CHO, unaltered CHO cells; DAF, CHO cells expressing full length DAF; Δ1-4, CHO cells expressing DAF with the corresponding SCR domain deleted. Note that OE-1 does not recognize SCR-3 domain deletion. Panel B represents 13 phage sequences and an overall consensus sequence eluted from 3 rounds of panning with OE-1-coupled sepharose. In panels B and C, OE-1 mAb was incubated with indicated concentrations of synthetic EVEHWYRSG peptide (SEQ ID NO: 36), or a control scrambled peptide (Scr), and used to probe Western blots from T84 epithelial lysates (panel C) or cell surface ELISA on intact T84 cells. Data are presented as mean±s.e.m. (n=3).

As shown in FIG. 6A, these studies revealed the loss of OE-1 reactivity in only the SCR-3 truncation. Such findings were not due to diminished overall DAF expression, as determined by blotting of total DAF content using polyclonal anti-DAF antisera. Such findings suggest that OE-1 recognizes an epitope encoded, at least in part, by the SCR-3 domain of DAF.

To better define the DAF epitope recognized by OE-1, we utilized the previously characterized LL9 phage library displaying linear 9-mer peptides to select for OE-1 binding epitopes (Mazzucchelli et al., 1999, Blood. 93: 1738-48). The library underwent 3 rounds of panning over bound OE-1 with intermediate amplifications. The round 3 enriched phage had a titer of 5.0×10⁹ PFU/ml and yielded an enrichment of greater than 3 logs relative to round 1. As shown in FIG. 6B, 13 phage clones were sequenced and aligned sequences revealed a number of structural similarities. In particular, a pattern amino acid sequence of EXEX*WX*RXX** (SEQ ID NO: 1, where X is neutral, X* is large and X** is hydrophobic) emerged as a relatively close consensus match. Based on our analysis of DAF, and consistent with our observations that OE-1 does not western blot under reducing conditions, this linear sequence did not exist in SCR domain 3. These findings suggest that OE-1 recognizes a non-linear constrained epitope where, at least in part, SCR-3 contributes to this binding.

As shown in FIG. 6, a synthetic peptide corresponding to a best-fit OE-1 epitope consensus (EVEHWYRSG, SEQ ID NO: 36), but not a scrambled peptide, blocked OE-1 binding to denatured protein (western blot, FIG. 6C) and to the intact epithelial cell surface protein (ELISA, FIG. 6D) in a concentration-dependent manner, suggesting that this EVEHWYRSG peptide (SEQ ID NO: 36) is a least a reasonable match for the OE-1 epitope. In it's labeled form (biotin-EVEHWYRSG), binding to the surface of intact PMN was readily detectable (fluorescence intensity 2,188±383 RFU, n=4) compared to either labeled control peptide (fluorescence intensity 81±12 RFU, p<0.01 compared to biotin-EVEHWYRSG) or to unlabeled peptide (fluorescence intensity 33±6 RFU, p<0.01 compared to biotin-EVEHWYRSG).

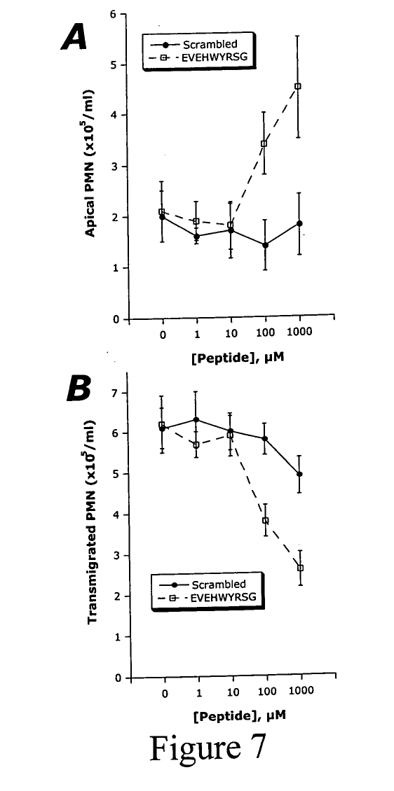

Next it was determined whether this synthetic peptide influenced the PMN transmigration and accumulation of PMN on the surface of epithelia following transmigration. The results of this experiement are shown in FIG. 7. PMN and the apical reservoir of epithelia were incubated with indicated peptides prior to co-incubation and assessment of transmigration. In panels A and B, the influence of indicated concentrations of OE-1 peptide EVEHWYRSG (SEQ ID NO: 36, dashed line), or a scrambled control (solid line), on the kinetics of transmigration was assessed. Panel A represents the number of PMN which remain attached to the apical epithelial membrane and panel B represents the number of PMN which completely traverse the epithelial monolayer. Data are presented as mean±s.e.m. (n=3).

As shown in FIG. 7, PMN and epithelial pre-exposure to increasing concentrations of EVEHWYRSG (SEQ ID NO: 36) but not scrambled peptide (SEQ ID NO: 35), resulted in both increased apical accumulation of PMN (FIG. 7A, p<0.025 by ANOVA) and diminished PMN transmigration (p<0.05 by ANOVA). Such data support our OE-1 findings and suggest that we can recapitulate this biology with a closely matched synthetic mimetic corresponding to the OE-1 epitope.

Next, the relative influence of individual DAF mimetic peptides was examined. To accomplish this, a series of four peptides was generated that contained alanine substitutions at conserved positions of the wild-type peptide (EVEHWYRSG, SEQ ID NO: 36). The sequence of individual synthetic peptides were (substituted alanine residues in bold underline):

| | | |
|---|---|---|
| Wild-type: | EVEHWYRSG | (SEQ ID NO: 36) |
| Peptide 1: | AVAHWYRSG | (SEQ ID NO: 37) |
| Peptide 2: | EVEHAYRSG | (SEQ ID NO: 38) |
| Peptide 3: | EVEHWARSG | (SEQ ID NO: 39) |
| Peptide 4: | EVEHWYASG | (SEQ ID NO: 40) |

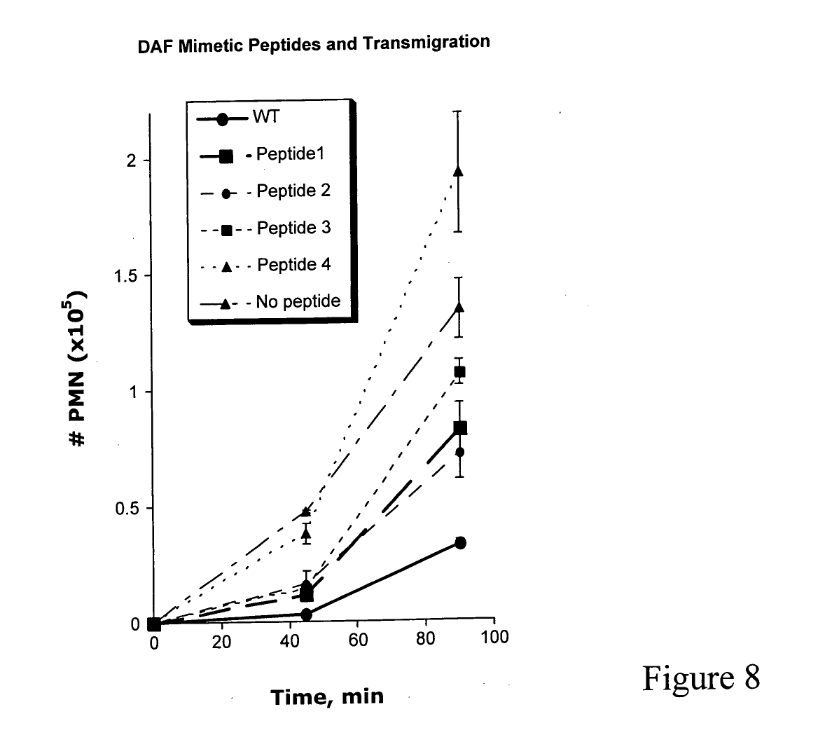

Next these alanine-substituted synthetic peptides were compared to the wild-type peptide in kinetic PMN transmigration assays, the results of which are shown in FIG. 8. PMN and the apical reservoir of epithelia were incubated with indicated peptides prior to co-incubation and assessment of transmigration. In panels A and B, the influence of indicated concentrations of OE-1 peptide EVEHWYRSG (SEQ ID NO: 36, dashed line), or a scrambled control (solid line), on the kinetics of transmigration was assessed. Panel A represents the number of PMN which remain attached to the apical epithelial membrane and panel B represents the number of PMN which completely traverse the epithelial monolayer. Data are presented as mean±s.e.m. (n=3).

As shown in FIG. 8, PMN and epithelial pre-exposure to individual peptides (Wild-type and Peptides 1-5, 1 mM final concentration) resulted in a rank order of potency Wild-type>Peptide 2=Peptide 1>Peptide 3>compared to no peptide controls (FIG. 8, p<0.025 by ANOVA). Peptide 4 appeared to enhance PMN transmigration relative to no peptide controls. Such data support the OE-1 findings and suggest that the original synthetic mimetic corresponding to the OE-1 epitope is potent and that individual amino acid substitutions have variable influences on biological activity.

### Example 5

### Relationship Between Hypoxia and DAF Expression and Regulation

Ongoing inflammatory responses are characterized by dramatic shifts in tissue metabolism. These changes include lactate accumulation with resultant metabolic acidosis and diminished availability of oxygen (hypoxia) (Kokura et al., 2002, Free Radic. Biol. Med. 33:427-32; Haddad, 2003, Crit. Care 7:47-54; Saadi et al., 2003, Faseb J. 16:849-56). Such shifts in tissue metabolism result, at least in part, from profound recruitment of inflammatory cell types, particularly myeloid cells such as neutrophils (PMN) and monocytes. The vast majority of inflammatory cells are recruited to, as opposed to being resident at, inflammatory lesions (Lewis et al., 1999, J. Leukoc. Biol. 66:889-900).

As such, it is important to understand the interactions between microenvironmental metabolic changes (e.g. hypoxia) as they relate to recruitment signals and molecular mechanisms utilized by myeloid cells during inflammation.

To examine the role of hypoxia in a model mucosal disease (inflammatory bowel disease IBD) the TNBS model of murine colitis was utilized. This decision was based on previous observations of inflammation-related disturbances of colonic blood flow in this model (Kruschewski et al., 2001, Dig Dis Sci. 46:2336-43), compatible with the functional and anatomical microvascular abnormalities that have been observed in human Crohn's disease patients (Wakefield et al., 1989, Lancet ii:1057-1062). Furthermore, close histological examination of colonic tissue derived from TNBS-colitic animals (7 days post induction), revealed a characteristically obliterating vasculitis of the small submucosal vessels in loose association to mucosal inflammation, which displayed similarities to observations in human specimen (Yokoyama et al., 2001, Hepatogastroenterology 48:401-7; Wakefield et al., 1989, Lancet ii:1057-1062).

To determine the extent of tissue hypoxia in this experimental model, the characteristic reduction and binding of the nitroimidazole compound EF5 to cellular macromolecules in absence of adequate oxygen levels (Evans et al., 2000, Cancer Res. 60:2018-24) was utilized. Colonic samples from vehicle-treated control animals revealed evident EF5 retention in superficial epithelial layers within the colon, consistent with reports suggesting that the intestine is relatively hypoxic compared to other mucosal tissues (Taylor et al., 1999, J. Biol. Chem. 274:19447-19450). In agreement with recent observations examining EF5 localization in the small intestine (Synnestvedt et al., 2002, J. Clin. Invest. 110:993-1002), such EF5 staining was almost exclusively localized to the epithelium. In contrast, animals exposed to the colitis-inducing hapten TNBS revealed a profound retention of EF5 within colonic epithelia. Moreover, such EF5 retention was prominently associated with colitic lesions both in superficial and in deeper submucosal regions of the mucosa. Such findings indicate that the TNBS-induced vascularity changes likely result in significant tissue hypoxia, predominantly within the epithelium.

Activation of Hypoxia-inducible Factor-1 (HIF-1) during colitis. Hypoxia-inducible factor (HIF) is a central regulatory transcription factor for hypoxia-induced gene expression, and serves as a sensitive and selective indicator of hypoxia (Becker et al., 2000, Am. J. Respir. Cell. Mol. Biol. 22:272-279). HIF-1 is a member of the Per-ARNT-Sim (PAS) family of basic helix-loop-helix (bHLH) transcription factors. HIF-1 activation is dependent upon stabilization of an O₂-dependent degradation domain of the α subunit and subsequent nuclear translocation to form a functional complex with HIF-1β and cofactors such as CBP and its ortholog p300 (Semenza, 2001, Cell 107:1-3). Under conditions of adequate oxygen supply, iron and oxygen dependent hydoxylation of two prolines (Pro564 and Pro 402) within the oxygen-dependent degradation domain (ODD) of HIF-1α initiates the association with the von Hippel-Lindau tumor suppressor protein (pVHL) and rapid degradation via ubiquitin-E3 ligase proteasomal targeting (Maxwell et al., 1999, Nature 399:271-5; Tanimoto et al., 2000, Embo J 19:4298-309). A second hypoxic switch operates in the carboxy terminal transactivation domain of HIF-1α. Here, hypoxia blocks the hydroxylation of asparagine-803 facilitating the recruitment of CBP/p300 (Lando et al., 2002, Science 295:858-861).

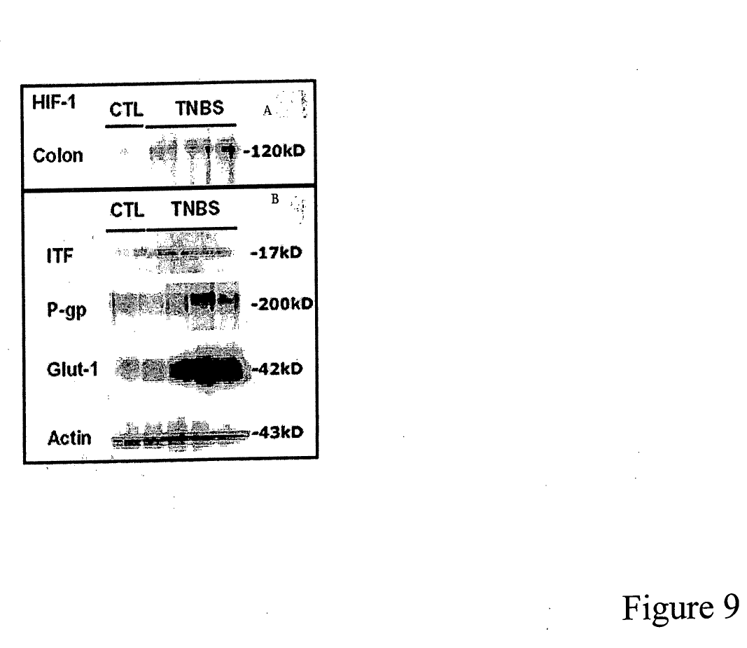

Based on the findings with EF5 localization, it was hypothesized that TNBS colitis results in HIF-1 activation, particularly within the epithelium. As regulation of HIF-1 occurs predominantly through stabilization of the α-subunit in response to hypoxia, the HIF-1α levels were analyzed in colonic tissue extracts derived from TNBS and vehicle control mice by western blot, shown herein in FIG. 9. FIG. 9A. demonstrates HIF-1α stabilization in TNBS colitis. Western blot analysis from TNBS and vehicle control animals 7 days after induction of colitis. Blots are derived from nuclear extracts from the colon of control and TNBS treated animals. In panel B, western blot analysis of the HIF-1 responsive genes intestinal trefoil factor (ITF), P-glycoprotein (P-gp) and glucose transporter-1 (Glut-1) were examined in control and TNBS treated animals.

As can be seen in FIG. 9A, TNBS colitis resulted in prominent HIF-1α expression compared to vehicle control. Such Hif-1α expression was evident by day 1 post-TNBS (data not shown) and most significant compared to controls at day 7. These findings were extended to determine whether HIF-1-regulated gene products were also induced in TNBS colitis. At day 7 post-induction, both the well characterized HIF-1 responsive gene product glucose transporter-1 (Glut-1) (Semenza, 1999, Ann. Rev. Cell. Dev. Biol. 15:551-78) and the more recently characterized barrier protective gene products ITF and the MDR1 gene product P-glycoprotein (P-gp) (Furuta et al., 2001, J. Ex. Med. 193:1027-1034; Comerford et al., 2002, Cancer Res. 62:3387-94) were prominently induced (FIG. 9B). These findings revealed correlative expression of HIF-1-regulated genes at time points where significant hypoxia and HIF-1 activation are evident.

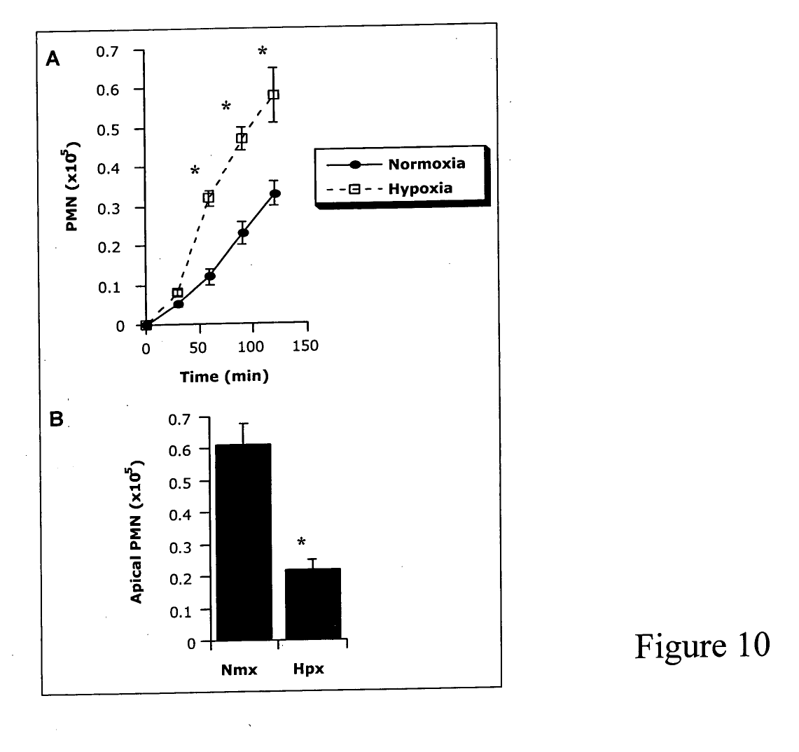

Increased PMN transmigration across intestinal epithelial cells by hypoxia. PMN trafficking into and across intestinal epithelia (e.g. the crypt abscess) is one of the pathological hallmarks of colitis (Zen et al., 2003, Curr Opin Cell Biol 15:557-64). Given this association between chronic inflammation (e.g. colitis) with epithelial hypoxia (see FIG. 9), we examined the molecular details of hypoxia-induced PMN-epithelial interactions. We have previously studied PMN transmigration following epithelial exposure to hypoxia (Synnestvedt et al., 2002, J. Clin. Invest. 110:993-1002; Colgan et al., 1996, J. Exp. Med. 184:1003-1015; Friedman et al., 1998, J. Cell. Physiol. 176:76-84; Eltzschig et al., 2003, J. Ex. Med. 198:783-796; Furuta et al., 2000, J. Leukoc. Biol. 68:251-9). Similar to these previous studies, the results shown in FIG. 10 revealed that epithelial pre-exposure to hypoxia increases PMN transmigration and apical accumulation of PMN. In panel A, Caco2 cells were pre-exposed to normoxia (closed circles) or hypoxia (open squares) for 48 hrs. Purified human PMN were applied to the basolateral chamber of transwell inverts. PMN were stimulated to migrate with FMLP in the physiologic direction (basolateral to apical) across the epithelial monolayer. Results are depicted as the number of transmigrated neutrophils over a 2 hr period. Results represent the mean±sem of three separate experiments, where * indicate significantly different than normoxia (p<0.01). In panel B, apical accumulation of human PMN is decreased following exposure of Caco2 cells to hypoxia. PMN accumulation at the apical surface of the intestinal epithelium is depicted for monolayers exposed to either normoxia (Nmx) or hypoxia (Hpx) for 48 hours. Results represent the mean±sem of three separate experiments, where * indicate significantly different than normoxia (p<0.01).

These previous studies implicated chemokines (such as IL-8) imprinting of the subepithelial matrix for initial recruitment of PMN to the basal pole of the epithelium (Furuta et al., 2000, J. Leukoc. Biol. 68:251-9), it was also clear that other factors, particularly cell surface protein(s) (Colgan et al., 1996, J. Exp. Med. 184:1003-1015), might also contribute to enhanced PMN transmigration.

Contribution of CD55 (DAF) to hypoxia-enhanced neutrophil trafficking across intestinal epithelia. Recently the regulation of CD55 by hypoxia was defined as it relates to neutrophil transmigration across intestinal epithelial cells in vitro. The basis of this observation stemmed from an initial screen of known neutrophil ligands expressed on intestinal epithelial cells (Zen et al., 2003, Curr Opin Cell Biol 15:557-64). Indeed, as shown in Table 1, mRNA expression patterns of PMN ligands in response to hypoxia were surprisingly stable. No differences in expression were observed for ICAM-1, occludin, JAM-A, JAM-B, or JAM-C were noted following 2, 6 or 18 hr hypoxia. A small decrease (˜30%) in CD47 expression was noted, although it was previously shown that CD47 is unlikely to be important for regulation of hypoxia-induced PMN transmigration (Colgan et al., 1996, J. Exp. Med. 184:1003-1015). The exceptional observation was CD55 (Table 1), whereby CD55 mRNA was rapidly (within 2 hrs) induced by hypoxia and returned to baseline within 18 hrs.

TABLE 1
Influence of Hypoxia on Epithelial
Expression of Putative PMN Ligands

| | 2 hours hypoxia, | 6 hours hypoxia, | 18 hours hypoxia, |
|---|---|---|---|
| Epithelial gene | fold change ± s.e. | fold change ± s.e. | fold change ± s.e. |
| DAF | 6.1 ± 0.29* | 2.4 ± 0.99* | 1.6 ± 0.03* |
| CD47 | 0.7 ± 0.06 | 0.6 ± 0.08 | 0.7 ± 0.07* |
| ICAM-1 | 0.9 ± 0.01 | 1.0 ± 0.003 | 0.9 ± 0.01 |
| Occludin | 1.0 ± 0.15 | 0.9 ± 0.26 | 1.1 ± 0.05 |
| JAM-1 | 1.1 ± 0.12 | 1.2 ± 0.05 | 0.9 ± 0.18 |
| JAM-2 | 0.9 ± 0.16 | 1.1 ± 0.11 | 0.9 ± 0.22 |
| JAM-3 | 0.9 ± 0.08 | 0.9 ± 0.09 | 0.9 ± 0.08 |

Fold change imRNA relative to normoxic controls, normalized for beta actin
*p < 0.025

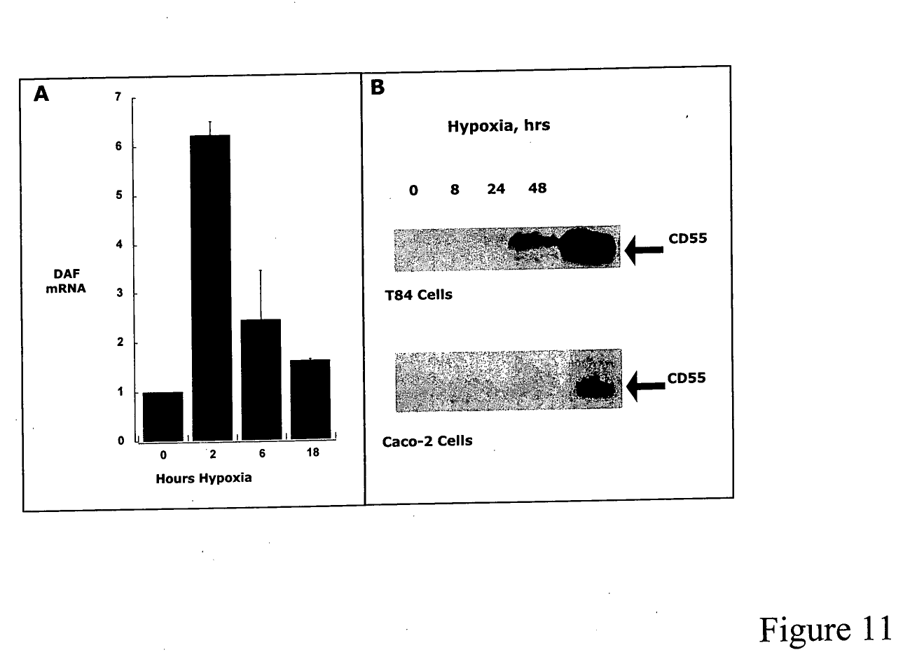

Next the relationship between CD55 mRNA and hypoxia was examined at the protein level. Panel A of FIG. 11 shows results using cultured T84 cell monolayers grown to confluence on membrane permeable supports. High resistance (TER>1000 ohm·cm²) were subjected to hypoxia for 0, 2, 6 or 18 hours of hypoxia. Relative CD55 mRNA levels were assessed by reverse transcription and real-time PCR with fold changes determined by normalization for background fluorescence and beta-actin expression relative to normoxic controls. Mean±s.e.m. from three separate determinations were obtained. In panel B, monolayers of T84 cells (top panel) or Caco-2 cells (bottom panel) were grown to confluence and exposed to 0, 8, 24, or 48 hours of hypoxia. Cells were lysed and resolved by SDS-PAGE. Resultant western blots were probed for CD55 using the mouse monoclonal anti-CD55 OE1 antibody. Results depict representative blots from 3 separate experiments for each cell line. CD55 protein expression was confirmed in both T84 and Caco-2 cells (FIG. 11B). Increased levels of CD55 protein (determined by western blot) were evident as early as 8 hours following hypoxic exposure and increased further relative to hypoxic controls by 24 hours hypoxia. Levels remained elevated relative to controls following 48 hours exposure. Caco-2 cells showed a somewhat delayed response relative to T84 cells with minimal induction in CD55 protein following 24 hours hypoxia but robust induction observed following 48 hours hypoxic exposure. Such findings identified that the apically expressed, epithelial anti-adhesive molecule CD55 as a dominantly expressed PMN ligand rapidly inducible by hypoxia.

Analysis of the CD55 gene promoter. The molecular details of CD55 induction by hypoxia in vitro were also studied. One approach profiled the CD55 promoter for transcription factor binding sites important for hypoxia regulation.

First, it was determined whether hypoxia-induced CD55 results from increased transcriptional activity. Caco2 cells were subjected to hypoxia in the presence or absence of the transcriptional inhibitor 5,6-dichlorobenzimidazole riboside (DRB, 10 μM) and assessed for changes in CD55 mRNA expression. These experiments revealed a 87±7% decrease in CD55 inducibility in the presence of DRB. Consequently, increases in CD55 by hypoxia required new mRNA synthesis.

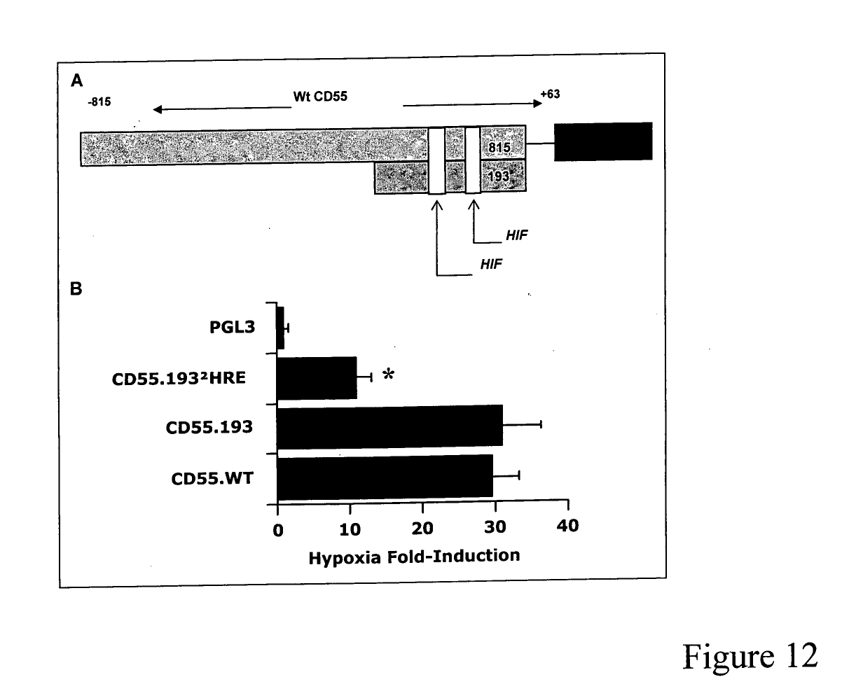

Second, insight was gained using CD55 promoter constructs. For such analysis, we cloned the CD55 promoter (Ewulonu et al., 1991, Proc. Natl. Acad. Sci. USA 88:4675-9) into a pGL3 luciferase reporter vector and screened this construct through transient transfection in Caco2 cells. As shown in FIG. 12, these studies revealed that, similar to the RNA and protein results, the wild-type CD55 promoter is hypoxia responsive (29±5-fold induction over normoxia, p<0.025). Truncation of this 815 bp promoter to 193 bp resulted in no loss of hypoxia activity (31±4-fold induction, p<0.025). Panel A represents the orientation of wild-type (WtCD55) and truncated CD55 (193 bp) luciferase reporter constructs. Also shown in the position of putative HIF and CREB binding sites. In panel B, Caco2 monolayers were transiently transfected with plasmids expressing sequence corresponding to full length CD55 (CD55.WT), truncated CD55 (CD55.193) or HIF-responsive element mutant (CD55.193ΔHRE) upstream from the luc reporter gene. Twelve hours later, cells were exposed to hypoxia or normoxia for 48 hrs and assessed for luciferase activity. All transfections were normalized to co-transfected Renilla activity. Data are mean±s.e.m. from three experiments where * indicates significantly different than WT or truncated CD55 (p<0.025).

Third, two putative hypoxia-responsive elements were studied within this 193 bp region (see map in FIG. 12). To date, we have mutated one of these sites (5′-CGAC-3′→5′-ATGA-3′ at positions −49 to −46 relative to the ATG) and revealed that this mutation, at least in part, results in decreased promoter activity (61±8% decrease in activity). Such results indicate that the CD55 promoter is hypoxia-responsive, and implicate HIF as a contributing factor to such regulation.

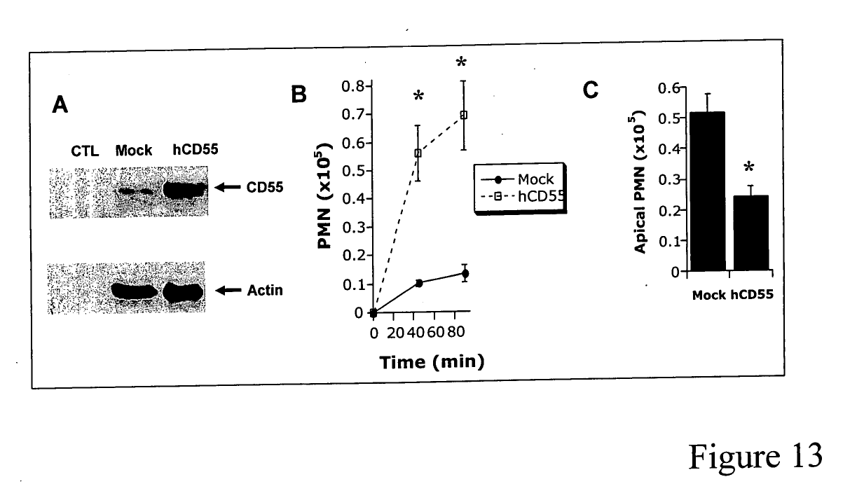

Over-expression of CD55 recapitulates hypoxia for functional regulation of PMN transmigration. Based on the above results, CD55 induction by hypoxia functionally explains increased PMN transmigration following hypoxia. To test this hypothesis, over-expression of CD55 was forced in intestinal epithelial cells under normoxic conditions, and the influence on PMN transmigration was examined. As shown in FIG. 13, transient transfection of full-length CD55 cDNA in Caco2 cells resulted in a nearly five-fold increase in CD55 expression, comparable to that seen with 48 hr hypoxia (see FIG. 11). Using these cells, next it was examined whether PMN transmigration, in normoxia, might be different than mock-transfected cells. As shown in FIG. 13, over-expression of CD55 resulted in a nearly 7-fold increase in PMN transmigration and a significant decrease in apical PMN. Panel A is a representative western blot depicting over-expression of CD55 by transient transfection with full-length CD55 cDNA, compared to mock-transfected cells. Actin was used to monitor equal loading (bottom). A no lysate control (CTL) is also shown. In panel B, human PMN were applied to the basolateral chamber of transwell inverts following either mock transfection or CD55 transfection of Caco2. PMN were stimulated to migrate with fMLP in the physiologic direction (basolateral to apical) across the epithelial monolayers. Results are depicted as the number of transmigrated neutrophils vs. time following 48 hours transfection. Results represent the mean±sem of three separate experiments, where * indicate significantly different than mock transfected (p<0.01). In panel C, apical accumulation of human PMN is decreased following transient transfection of Caco2 cells with full-length human CD55. PMN accumulation at the apical surface of the intestinal epithelium is depicted for monolayers transfected for 48 hours, compared to mock transfected controls. Results represent the mean±sem of three separate experiments, where * indicate significantly different than mock transfected (p<0.01). This pattern is precisely the pattern that was observed following epithelial exposure to hypoxia (see FIG. 10).

From these results it was concluded that it is likely that DAF induction by microenvironmental conditions found within inflammatory foci (i.e. hypoxia) represents a pathway for enhanced leukocyte trafficking within chronically inflamed mucosal tissues. Moreover, the identification of HIF-regulated CD55 provides a new target for development of novel anti-inflammatory therapies directed against DAF. For example, an agent that interferes with inflammation (e.g. interferes with neutrophil transmigration) can be identified by contacting a cell expressing DAF and/or HIF (or an in vitro expression system) with a candidate agent and assaying for a decrease in DAF and/or HIF expression and/or activity (DAF expression/activity can be used to monitor HIF expression/activity) relative to a control.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

The content of each patent and scientific publication, and any other reference listed herein, is hereby incorporated by reference in its entirety to the extent that it contains relevant technical information.

1. An isolated polypeptide that binds decay accelerating factor (DAF) and competitively inhibits the specific binding of OE-1 antibody to DAF.

2. The isolated polypeptide of claim 1, wherein the isolated polypeptide binds SCR-3 epitope of DAF.

3. The isolated polypeptide of claim 1, wherein the polypeptide is an antibody or antigen binding fragment thereof.

4. The isolated polypeptide of claim 3, wherein the antibody or antigen-binding fragment thereof interferes with transmigration of neutrophils across a cellular membrane.

5. The isolated polypeptide of claim 3, wherein the antibody or antigen-binding fragment thereof is selected for its ability to bind living cells.

6. The isolated polypeptide of claim 3, wherein the antibody or antigen-binding fragment thereof binds to a conformational epitope of DAF.

7. The isolated polypeptide of claim 3, wherein the antibody is a monoclonal antibody.

8. The isolated polypeptide of claim 7, wherein the monoclonal antibody is OE-1.

9. The polypeptide of claim 3, wherein the antibody or antigen-binding fragment thereof binds SCR-3 epitope of DAF.

10. An isolated polypeptide which has the amino acid sequence EX₁EX₂WX₂R X₁X₃ (SEQ ID NO: 1), wherein X₁ is a neutral amino acid, X₂ is a large amino acid and X₃ is a hydrophobic amino acid.

11-19. (canceled)

20. A recombinant expression vector comprising an isolated nucleic acid operably-linked to a promoter, wherein the isolated nucleic acid encodes the polypeptide of claim 10 or a complement thereof.

21-27. (canceled)

28. A method for modulating an immunological interaction comprising contacting cells bearing DAF with an agent that binds DAF and that interferes with the interaction between neutrophils and the cells bearing DAF.

29-32. (canceled)

33. A method for treating inflammation comprising administering to a subject in need of such treatment an effective amount of an agent that binds DAF or mimics SCR-3 epitope of DAF, wherein the agent interferes with the interaction between neutrophils and DAF bearing cells.

34-38. (canceled)

39. A method for interfering with neutrophil transmigration comprising,
contacting a barrier across which neutrophils transmigrate and including cells expressing DAF with an agent that binds DAF.

40-44. (canceled)

45. A method for identifying an agent that interferes with transmigration of neutrophils comprising,
contacting a membrane across which said neutrophils transmigrate with an agent that binds DAF,
contacting the membrane with said neutrophils, and
determining whether the neutrophils transmigrate across the membrane.

46-53. (canceled)
